(19) United States
(12) Patent Application Publication
(10) Pub. No.: US 2022/0354791 A1
(43) Pub. Date: Nov. 10, 2022

(54) MESOPOROUS POLYMERIC PARTICULATE MATERIAL

(71) Applicant: Aston University, Birmingham West Midlands (GB)

(72) Inventors: Ali AL-KHATTAWI, Birmingham West Midlands (GB); Tuan-Tu LE, Birmingham West Midlands (GB)

(21) Appl. No.: 17/621,495

(22) PCT Filed: Jun. 24, 2020
(86) PCT No.: PCT/EP2020/067686

§ 371 Date: Dec. 21, 2021

(30) Foreign Application Priority Data
Jun. 25, 2019 (GB) 1909137.0

(51) Int. Cl. A61K 9/16; C08J 9/28; A61K 31/4422; A61K 31/192; A61K 31/635; A61K 49/00

(52) U.S. Cl. CPC A61K 9/1652; C08J 9/283; A61K 31/4422; A61K 31/192; A61K 31/635; A61K 49/0093; A61K 49/0043; C08J 2201/0504; C08J 2201/0502; C08J 2207/10; C08J 2205/042; C08J 2301/14; C08J 2301/12; C08J 2301/28

(57) ABSTRACT

A particulate material comprising porous polymeric particles is described. The porous polymeric particles have an average pore diameter of from 2 to 50 nm and a volume mean particle diameter D[4,3] of less than 100 μm. The material is obtained or obtainable by spray-drying a polymer solution. The particles find use as a solubility-enhancing carrier for active pharmaceutical compounds. Methods of manufacturing the particulate material and pharmaceutical compositions including the particulate material loaded with one or more active pharmaceutical compounds are also described.

### RELATED APPLICATION

This application is related to and claims priority from United Kingdom patent application number 1909137.0 filed 25 Jun. 2019, the contents of which are incorporated herein by reference in their entirety.

### FIELD OF THE INVENTION

The present invention relates to a porous particle and particularly, although not exclusively, to a mesoporous particle for use as a carrier or sorbent for drug compounds to enhance the solubility of such compounds and/or provide an extended or sustained release pharmaceutical composition.

### BACKGROUND

According to US Food and Drug Administration's guidance for industry (2017), drugs can be classified into one of four categories of the Biopharmaceutics Classification System (BCS): high solubility, high permeability (BCS I); low solubility, high permeability (BCS II); high solubility, low permeability (BCS III); and low solubility and low permeability (BCS IV). A drug substance is considered as “poorly soluble drug” when the highest dose is not soluble in 250 mL of aqueous media within the pH range of 1.0 to 6.8, e.g. 0.1 N HCl or simulated gastric fluid without enzymes; a pH 4.5 buffer; a pH 6.8 buffer or simulated intestinal fluid without enzymes, at temperature of 37±1° C.

Following oral administration, drugs must dissolve in gastrointestinal fluids in order to be absorbed across the intestinal mucosa into the systemic circulation and exert a therapeutic action. Formulation development of low solubility drugs (BCS II and IV) faces great challenge as these drugs are poorly absorbed and usually exhibit subsequent low and variable oral bioavailability (Bosselmann & “Route-Specific Challenges in the Delivery of Poorly Water-Soluble Drugs”, Formulating poorly soluble drugs, 2012, pp. 1-26). Over 40% of drugs on the market are in BCS Class II or IV which have low solubility. New chemical entities are even less soluble compared to marketed products with a projection of up to 70-90% of drug candidates in the pipeline suffering from low solubility (Ting et al., “Advances in Polymer Design for Enhancing Oral Drug Solubility and Delivery”, Bioconjugate Chem, 2018, 29, pp. 939-952).

The problem of low solubility has so far been addressed using solubilisation techniques, including solid dispersion systems, size reduction, salt formation, use of more highly soluble prodrugs, and the use of liposomes. Among these techniques, solid dispersion systems are likely to be increasingly utilised for enhanced solubility of poorly soluble drugs. Solid dispersion systems are mainly based on a so-called “amorphisation” effect whereby the crystalline drugs are converted into their amorphous form when adsorbed onto the solid carrier, which exhibits superior solubility in comparison with that of the original crystalline form. Mesoporous materials (porous materials having an average pore diameter of 2 to 50 nm) are considered as highly effective carriers for drug amorphisation due to tuneable pore size and high surface area. Furthermore, this approach is widely applicable for existing poorly soluble drugs and drug candidates in the pipeline with various chemical structures (Ibid. Bosselmann & Williams; Choudhari et al., 2014, “Mesoporous Silica Drug Delivery Systems”, Amorphous Solid Dispersions—Theory and Practice, pp. 665-693; Laitinen et al., 2014, “Theoretical Considerations in Developing Amorphous Solid Dispersions”, Amorphous Solid Dispersions—Theory and Practice, pp. 35-90; Riikonen et al., 2018. “Mesoporous systems for poorly soluble drugs—recent trends”, International Journal of Pharmaceutics, 536 (1), 178-186).

The spatial confinement of the drug molecules within the nanometre-scale pores prevents drug recrystallization and maintains the amorphous state of the drug. As a result of this, a significant enhancement of drug solubility and subsequent dissolution could be achieved due to the attainment of a highly soluble amorphous form (Garcia-Bennett A., Feiler, A., 2014, “Mesoporous ASD: Fundamentals”, Amorphous Solid Dispersions—Theory and Practice, pp. 637-663; Shen et al., 2017, “Mesoporous materials and technologies for development of oral medicine”, Nanostructures for Oral Medicine, pp. 699-749).

Current mesoporous materials used for drug delivery purposes are based on mesoporous silica, discovered at Mobil Corporation (Kresge et al., 1992, “Ordered mesoporous molecular sieves synthesized by a liquid-crystal template mechanism”, Nature, 359, pp. 710-712), and include Silsol® and Syloid® marketed by W.R. Grace & Co. The synthesis of mesoporous materials mainly utilises templating agents (pore forming agents) such as surfactant templates, crystal templates, polymeric templates or emulsion templates to form mesopores in the resulting solid materials. Using templating agents to facilitate the pore formation has been proven successful to produce various mesoporous materials such as mesoporous silica and aluminium. However, there is a considerable issue as the process using templating agents is complicated and time-consuming due to high temperature post-treatment (Nandiyanto & Okuyama, 2011, “Progress in developing spray-drying methods for the production of controlled morphology particles: From the nanometre to submicrometer size ranges”, Advanced Powder Technology, 22, pp. 1-19). For at least this reason the current market value of mesoporous silica materials for drug delivery applications is in the region of thousands of Euros per kg, which greatly increases the cost of the finished dosage form after drug loading. Inorganic mesoporous materials such as mesoporous silica also suffer from the presence of impurities such as inherent trace metals and strong alkaline/acidic residues which potentially cause drug stability issues.

Furthermore, although existing mesoporous materials provide increased solubility of drug molecules through amorphisation, the corollary is that the drug is rapidly released from the mesoporous carrier in the body after the composition has been ingested. Applications are thereby limited to long half-life drugs, or frequent administration is required when short half-life drugs are incorporated, which leads to problems with patient compliance.

There is a need for improved drug delivery vehicles which offer enhanced solubility for poorly soluble drugs and vehicles which may provide further benefits such as compatibility with short half-life drugs.

The present invention has been devised in the light of the above considerations.

### SUMMARY OF THE INVENTION

At its most general, the present invention relates to porous particles for use as a carrier or sorbent for drug compounds.

According to a first aspect of the present invention, there is provided a particulate material comprising porous polymeric particles, the average pore diameter being from 2 to 50 nm, wherein the porous polymeric particles have a volume mean particle diameter D[4,3] of less than 100 μm and the material is obtained or obtainable by spray-drying a polymer solution.

In some embodiments, there is provided a particulate material comprising porous polymeric particles, the porous polymeric particles comprising a plurality of pores having an average pore diameter of from 2 to 50 nm, wherein the porous polymeric particles have a volume mean particle diameter D[4,3] of less than 100 μm and the material is obtained or obtainable by spray-drying a polymer solution.

Porous polymeric particles, more specifically mesoporous polymeric particles comprising pores having an average pore diameter of from 2 to 50 nm, are produced by spray drying. The pores are of a size which facilitates the adsorption and amorphisation of a wide range of active pharmaceutical compounds (drug compounds), thereby improving the solubility of the compounds by converting them from a less soluble crystalline phase into a more soluble amorphous phase when adsorbed within the pores of the particle.

The invention is therefore particularly applicable to poorly soluble drug compounds, the solubility of which may be enhanced by adsorbing the compound onto the polymeric particle of the invention. For certain drug compounds the particulate material may provide around a ten-fold increase in apparent solubility of the compound loaded onto the particle versus the free compound. The polymeric particles may be produced by straightforward spray-drying procedures without the need for templating agents, surfactants or other complex manufacturing or purifying techniques, thereby provide a low-cost alternative to existing inorganic mesoporous materials such as mesoporous silica. Additionally the polymeric particles of the invention, unlike inorganic mesoporous materials, do not contain any trace metals or strong alkaline/acidic residues which would compromise drug stability.

According to a second aspect of the present invention, there is provided a pharmaceutical composition comprising a particulate material according to the first aspect loaded with one or more active pharmaceutical compounds.

A third aspect of the invention is a pharmaceutical composition according to the second aspect, for use in therapy.

A fourth aspect of the invention is a method of treatment of the human or animal body, comprising administration of a therapeutically effective amount of a pharmaceutical composition according to the second aspect to a patient in need thereof.

A fifth aspect of the invention is a method of manufacturing a particulate material comprising spray-drying a polymer solution, the particulate material comprising porous polymeric particles, the average pore diameter being from 2 to 50 nm, wherein the porous polymeric particles have a volume mean diameter D[4,3] of less than 100 μm.

A sixth aspect of the invention is the use of a particulate material according to the first aspect as a solubility-enhancing carrier for one or more active pharmaceutical compounds.

According to another aspect of the present invention, there is provided a particulate material comprising porous polymeric particles, the average pore diameter being from 2 to 50 nm, wherein the porous polymeric particles have a volume mean particle diameter D[4,3] of less than 100 μm. In some embodiments the material is obtained or obtainable by spray-drying a polymer solution.

As used herein, the term “porous” denotes a particle which contains open pores at the surface of the particle. The particle may also contain additional pores as part of a network of pores through the bulk of the particle. The term “mesoporous” denotes a particle which contains surface pores having an average pore diameter of from 2 to 50 nm (according to the IUPAC definition).

As used herein, the term “average pore diameter” denotes the mean average pore diameter as measured by gas adsorption porosimetry under the BJH (Barrett-Joyner-Halenda) theory, for example using a pore size analyser such as Quantachrome Nova 4200e, (e.g. according to the method in ISO 15901-2 of 2006—“Pore size distribution and porosity of solid materials by mercury porosimetry and gas adsorption—Part 2: Analysis of mesopores and macropores by gas adsorption”). The average pore diameter herein is calculated from the total pore volume and specific surface area by assuming that pore geometry is cylindrical. The total pore volume may be estimated from the nitrogen amount adsorbed at a relative pressure P/Po of 0.95 by assuming that all the pores are then filled with liquid nitrogen. The specific surface area may be determined by Brunauer-Emmett-Teller (BET) method (Quantachrome instruments, 2009, Nova operation manual version 11.02).

For example, assuming cylindrical pore geometry, the average pore diameter can be expressed as

$$Average pore size = \frac{4 V}{S}$$

where V is the volume of liquid nitrogen contained in the pores and S is the specific surface area of porous polymeric particles.

The term “poorly soluble” herein is used generally to encompass the terms “sparingly soluble”, “slightly soluble”, “very slightly soluble” and “practically insoluble”, which are defined in the section Solubility—Part III—General Notices of British Pharmacopoeia (BP) 2019 and European Pharmacopoeia (EP) 9^{th}, as follows:

Sparingly soluble: 30-100 mL of aqueous medium is required to dissolve 1 g of substance at a temperature between 15 and 25° C.

Slightly soluble: 100-1000 mL of aqueous medium is required to dissolve 1 g of substance at a temperature between 15 and 25° C.

Very slightly soluble: 1000-10,000 mL of aqueous medium is required to dissolve 1 g of substance at a temperature between 15 and 25° C.

Practically insoluble: >10,000 mL of aqueous medium is required to dissolve 1 g of substance at a temperature between 15 and 25° C.

A first aspect of the invention is a particulate material comprising porous polymeric particles, more specifically mesoporous polymeric particles.

The porous polymeric particles have a volume mean particle diameter, D[4,3] (also denoted D_{4,3}), of less than 100 μm. In some embodiments, D[4,3] is less than 95 μm, for example less than 90 μm, less than 85 μm, less than 80 μm, less than 75 μm, less than 70 μm, less than 65 μm, less than 60 μm, less than 55 μm or less than 50 μm. D[4,3] may be measured by techniques known to the skilled person, such as laser diffraction techniques using the method in ISO 13320 of 2009, for example using a Malvern Mastersizer 3000.

In some embodiments, the porous polymeric particles have a D[4,3] of at least 5 μm, for example at least 10 μm, at least 15 μm, at least 16 μm, at least 17 μm, at least 18 μm, at least 19 μm or at least 20 μm. In some embodiments, the particle has a D[4,3] of from 5 to 100 μm, for example from 5 to 90 μm, from 5 to 80 μm, from 10 to 80 μm, from 10 to 70 μm, from 15 to 70 μm, from 15 to 60 μm or from 20 to 60 μm.

There are a number of important parameters which can be used to describe porosity properties of porous solids such as specific surface area, pore volume, average pore diameter, and pore size distribution (Recommendations for the Characterization of Porous Solids, Pure & Appl. Chem., Vol. 66, No. 8, pp. 1739-1758, 1994).

The particles of the material have a mean average pore diameter (e.g. average surface pore diameter) of from 2 to 50 nm, as measured by gas adsorption porosimetry under the BJH (Barrett-Joyner-Halenda) theory, for example using a pore size analyser such as Quantachrome Nova 4200e, (e.g. according to the method in ISO 15901-2 of 2006—“Pore size distribution and porosity of solid materials by mercury porosimetry and gas adsorption—Part 2: Analysis of mesopores and macropores by gas adsorption”). As explained above, assuming cylindrical pore geometry, the average pore diameter can be expressed as

$$Average pore size = \frac{4 V}{S}$$

where V is the volume of liquid nitrogen contained in the pores and S is the specific surface area of porous polymeric particles determined according to the BET theory.

In some embodiments, the average pore diameter is from 2 to 45 nm, for example from 2 to 40 nm, from 2 to 35 nm, from 2 to 30 nm, from 5 to 45 nm, from 5 to 40 nm, from 5 to 35 nm, from 5 to 30 nm, from 10 to 45 nm, from 10 to 40 nm, from 10 to 35 nm or from 10 to 30 nm.

The volume of the pores in the particles of the material (e.g. surface pore volume) may be greater than 0.10 cm³/g, for example greater than 0.15 cm³/g, greater than 0.20 cm³/g, greater than 0.25 cm³/g or greater than 0.30 cm³/g. In some embodiments the volume of pores may be from 0.10 to 0.50 cm³/g, for example from 0.10 to 0.45 cm³/g, from 0.10 to 0.40 cm³/g, from 0.15 to 0.45 cm³/g, from 0.15 to 0.40 cm³/g, from 0.20 to 0.45 cm³/g, from 0.20 to 0.40 cm³/g or from 0.25 to 0.40 cm³/g. Pore volume may be measured using the same techniques as used to measure average pore diameter, namely gas adsorption porosimetry under the BJH (Barrett-Joyner-Halenda) theory, for example using a pore size analyser such as Quantachrome Nova 4200e, (e.g. according to the method in ISO 15901-2 of 2006—“Pore size distribution and porosity of solid materials by mercury porosimetry and gas adsorption—Part 2: Analysis of mesopores and macropores by gas adsorption”).

In some embodiments, the material has a specific surface area greater than 10 m²/g, for example greater than 15 m²/g, greater than 20 m²/g, greater than 25 m²/g, greater than 30 m²/g, greater than 35 m²/g or greater than 40 m²/g. In some embodiments, the material has a specific surface area of up to 70 m²/g, for example up to 65 m²/g, up to 60 m²/g, up to 55 m²/g or up to 50 m²/g. In some embodiments, the material has a specific surface area of from 10 to 70 m²/g, for example from 15 to 70 m²/g, 15 to 65 m²/g, 15 to 60 m²/g, 20 to 60 m²/g, 20 to 55 m²/g, 25 to 55 m²/g, 30 to 55 m²/g, 35 to 60 m²/g, 35 to 55 m²/g or 40 to 50 m²/g. The specific surface area may be measured using the same techniques as used to measure average pore diameter, namely gas adsorption porosimetry under the BET (Brunauer-Emmett-Teller) theory, for example using a pore size analyser such as Quantachrome Nova 4200e, (e.g. according to the method in ISO 9277 of 2010).

Pore size distribution is the distribution of pore volume with respect to pore size (IUPAC Compendium of Chemical Terminology, 2014). Mesopore size calculations are performed using the method of Barrett, Joyner and Halenda (BJH) using the Kelvin model of pore filling starting from the Kelvin equation:

$$\frac{1}{{r}_{1}} + \frac{1}{{r}_{2}} = - \frac{RT}{{σ}^{{lg}_{{v}^{1}}}} ln ( \frac{p}{{p}^{0}} )$$

where R is the universal gas constant, T is temperature, r₁ and r₂ are the principal radii of curvature of the liquid meniscus in the pore, (p/p⁰) is the relative pressure at which condensation occurs, σ^{lg} is the surface tension of the liquid condensate and v¹ is its molar volume. This approach may be used to determine pore diameter, assuming a model for the pore shape in which the pores are cylindrical and the meniscus is hemispherical (r₁=r₂).

Rearrangement of the Kelvin equation and replacement of the principal radii of curvature terms by 2/r_{K} gives:

$${r}_{K} = \frac{2 {σ}^{lg} {v}^{1}}{RT ln ( \frac{{p}^{0}}{p} )}$$

where r_{K} is often referred to as the Kelvin radius.

If the pore radius of a cylindrical pore is rₚ and a correction is made for the thickness of a layer already adsorbed on the pore walls:

rₚ=r_{K}+2t

So, the pore diameter D is given by:

D=r_{K}+t

The pore size distribution (distribution of pore volume with respect to pore size) is usually represented graphically as dV/dD versus D, i.e. a plot of differential pore volume on the y-axis versus pore diameter on the x-axis. In cases where the variation of particle diameter is large, the y-axis variable may be replaced by dV/d(log D). The unit for dV/dD is (cm³/g)/nm and it represents the pore volume density. For a plot of dV/dD versus D, the peak area under the curve between any two pore sizes is proportional to the partial specific pore volume for the specific pore size interval.

Determination of pore volume, pore diameter and pore size distribution under the BJH theory may be made according to the method in ISO 15901-2 of 2006—“Pore size distribution and porosity of solid materials by mercury porosimetry and gas adsorption—Part 2: Analysis of mesopores and macropores by gas adsorption”.

In some embodiments, the material has a pore size distribution of from 0.5 to 100 nm, for example from 0.5 to 95 nm, from 0.5 to 90 nm, from 0.5 to 85 nm, from 0.5 to 80 nm, from 1 to 100 nm, from 1 to 95 nm, from 1 to 90 nm, from 1 to 85 nm, from 1 to 80 nm, from 1 to 75 nm, from 1 to 70 nm, from 2 to 100 nm, from 2 to 95 nm, from 2 to 90 nm, from 2 to 85 nm, from 2 to 80 nm, from 2 to 75 nm or from 2 to 70 nm. That is to say, the pores may have diameters falling within one of the above ranges.

The properties of the pores of the particle described herein, such as pore volume, average pore diameter and pore size distribution relate to surface pores (i.e. open pores at the surface of the particles within the material). The particles may nevertheless also contain internal (closed or open) pores formed during the spray-drying process, but the skilled person will understand that such internal closed pores cannot be measured using surface analysis techniques such as BET or BJH analysis.

In some embodiments, the porous polymeric particles comprise both internal and external pores, which may be confirmed for example by evaluation of SEM images. Without wishing to be bound by theory, it is believed that external (surface) pores act as a gateway for drug species to pass through and migrate into the interior of the particles during a drug-loading process, and to facilitate release of the drug from the particle upon contact with biological fluid. It is also believed that the presence of an internal mesoporous network enhances the “amorphisation” effect wherein crystalline drug compounds are converted into their high-energy amorphous form which exhibits superior solubility in comparison with the lower energy crystalline form.

The particles are polymeric particles, i.e. particles which comprise or consist of one or more polymeric materials. In some embodiments, the particles comprise or consist of one or more biocompatible polymeric materials, that is to say polymeric materials which have been approved for medical applications. In some embodiments, the particles comprise or consist of one or more cellulosic polymers. A cellulosic polymer is a polymer which is a derivative of cellulose, for example a polymer obtained by the chemical modification of the side chains of cellulose. In some embodiments the cellulosic polymer is selected from one or more of cellulose esters and cellulose ethers. In some embodiments, the cellulosic polymer is selected from one or more of cellulose acetate butyrate, cellulose acetate, methyl cellulose, ethyl cellulose, hydroxyethyl cellulose, hydroxypropyl cellulose, carboxymethyl cellulose and hydroxypropyl methyl cellulose. In some embodiments, the cellulosic polymer is selected from one or more of cellulose acetate butyrate, cellulose acetate, ethyl cellulose and hydroxypropyl cellulose. In some embodiments, the particles comprise or consist of cellulose acetate butyrate. In some embodiments, the particles comprise or consist of ethyl cellulose. Cellulosic polymers are preferred due to their biocompatibility, which makes them safe for in vivo administration, and high glass transition temperature, which facilitates pore formation.

In some embodiments, the particles comprise a single type of polymer. In some embodiments, the particles comprise a single type of polymer and the polymer is a derivative of cellulose. In some embodiments, the particles comprise a single type of polymer and the polymer is selected from cellulose esters and cellulose ethers. In some embodiments, the particles comprise a single type of polymer and the polymer is selected from cellulose acetate butyrate, cellulose acetate, methyl cellulose, ethyl cellulose, hydroxyethyl cellulose, hydroxypropyl cellulose, carboxymethyl cellulose and hydroxypropyl methyl cellulose. In some embodiments, the particles comprise a single type of polymer and the polymer is selected from cellulose acetate butyrate, cellulose acetate, ethyl cellulose and hydroxypropyl cellulose.

In other embodiments, the particles comprise two or more different types of polymer. In some embodiments, the particles comprise two different types of polymer. In some embodiments, the particles comprise two or more different types of polymer which are each independently selected from derivatives of cellulose. In some embodiments, the particles comprise two or more different types of polymer which are each independently selected from cellulose esters and cellulose ethers. In some embodiments, the particles comprise two or more different types of polymer which are each independently selected from cellulose acetate butyrate, cellulose acetate, methyl cellulose, ethyl cellulose, hydroxyethyl cellulose, hydroxypropyl cellulose, carboxymethyl cellulose and hydroxypropyl methyl cellulose. In some embodiments, the particles comprise two or more different types of polymer which are each independently selected from cellulose acetate butyrate, cellulose acetate, ethyl cellulose and hydroxypropyl cellulose. In some embodiments, the particles comprise two different types of polymer wherein a first polymer is ethyl cellulose and a second polymer is cellulose acetate butyrate.

Including two or more different types of polymer in the solution to be spray-dried and thereby in the final polymeric particles allows the properties of the particles, such as pore morphology, to be tailored by varying the relative quantities of the two or more polymers.

As will be understood by the skilled person, the polymer in the solution which is spray-dried to create the porous particles will be the same polymer which forms the porous particles themselves, so any discussion herein of the nature of the polymer in the particles applies equally to the polymer in the solution, and vice versa.

In some embodiments, the particles comprise or consist of cellulose acetate butyrate having a butyryl content of from 15 to 50 wt %, an acetyl content of from 1 to 30 wt % and a hydroxyl content of from 0.5 to 5 wt %. Suitable cellulose acetate butyrate polymers are known to the skilled person and commercially available, for example from Eastman Chemical.

In some embodiments, the polymer has a glass transition temperature (T_{g}) of at least 60° C., or greater than 60° C. In some embodiments, the polymer has a glass transition temperature of at least 65° C., for example at least 70° C., at least 75° C., at least 80° C., at least 85° C., at least 90° C., at least 95° C. or at least 100° C., for example greater than 100° C. In some embodiments, the polymer has a glass transition temperature of from 60 to 200° C., for example from 65 to 200° C., 70 to 200° C., 75 to 200° C., 80 to 200° C., 85 to 200° C., 90 to 200° C., 100 to 200° C., 100 to 195° C., 100 to 190° C., 100 to 185° C., 100 to 180° C., 100 to 175° C., 100 to 170° C. or 100 to 165° C. Such glass transition temperatures are preferred because they provide thermally stable polymers which can endure high temperature and allow the diffusion of solvents out of the polymer during spray drying without modifying the internal pore structure.

In some embodiments, the inlet temperature during spray-drying is lower than the glass transition temperature T_{g} of the polymer in the polymer solution. In this way, pore formation is facilitated and the formation of mesopores of the correct size is promoted. A polymer with a T_{g} higher than the inlet temperature has good thermal stability and can endure high temperature and allow the diffusion of solvents out of the polymer without modifying the internal pore structure. In some embodiments, the polymer has a glass transition temperature (T_{g}) of at least 100° C., or greater than 100° C., since this provides for a more flexible spray-dryer inlet temperature while ensuring that the inlet temperature remains below T_{g} of the polymer (i.e. the inlet temperature of the spray-dryer may be at least up to 100° C.).

In some embodiments, the weight average molecular weight (M_{w}) of the polymer is from 10,000 to 1,000,000 g/mol, for example from 20,000 to 900,000 g/mol, from 25,000 to 800,000 g/mol, from 30,000 to 700,000 g/mol, from 40,000 to 600,000 g/mol or from 50,000 to 500,000 g/mol.

In some embodiments, the number average molecular weight (Mₙ) of the polymer is from 5,000 to 500,000 g/mol, for example from 6,000 to 450,000 g/mol, from 7,000 to 400,000 g/mol, from 8,000 to 300,000 g/mol, from 9,000 to 200,000 g/mol or from 10,000 to 100,000 g/mol.

In some embodiments, the polymer is ethyl cellulose with an M_{w} of from 90,000 to 450,000.

In some embodiments, the polymer is cellulose acetate with an Mₙ of from 30,000 to 40,000.

In some embodiments, the polymer is cellulose acetate butyrate with an Mₙ of from 30,000 to 70,000.

In some embodiments, the polymer is a cellulose derivative polymer and has a viscosity of from 0.35 to 120 cps as measured by ASTM D1343 (Standard Test Method for Viscosity of Cellulose Derivatives by Ball-Drop).

In some embodiments, the polymer is a cellulose derivative polymer and has a viscosity of from 0.35 to 120 cps as measured by ASTM D1343 (Standard Test Method for Viscosity of Cellulose Derivatives by Ball-Drop) and a glass transition temperature of at least 60° C.

In some embodiments, the polymer is a cellulose derivative polymer and has a viscosity of from 0.35 to 120 cps as measured by ASTM D1343 (Standard Test Method for Viscosity of Cellulose Derivatives by Ball-Drop), and the inlet temperature during spray-drying is lower than the glass transition temperature T_{g} of the polymer.

Another aspect of the invention is a pharmaceutical composition comprising a particulate material according to the first aspect and one or more active pharmaceutical compounds. In some embodiments, one or more active pharmaceutical compounds are adsorbed onto the surface of the particles, including within the surface pores. In some embodiments, one or more active pharmaceutical compounds are contained within the internal structure of the particle, for example within an internal pore, by providing a solution for spray-drying which contains both the polymer and the one or more active pharmaceutical compounds. This may provide a pharmaceutical composition which provides extended (or sustained) release of the one or more active pharmaceutical compounds in vivo after ingestion of the composition, since the one or more active pharmaceutical compounds are at least partially entrapped within the structure of the particle which prevents immediate release.

Because the surface pores of the particles have an average pore diameter of from 2 to 50 nm, the one or more active pharmaceutical compounds adsorbed to the surface of the particles within the pores are in an amorphous phase (i.e. they are amorphised), increasing the solubility of the one or more active pharmaceutical compounds. The material of the invention thereby provides a means to enhance the solubility of active pharmaceutical compounds, for example enhancing the solubility of poorly soluble active pharmaceutical compounds.

In some embodiments, the active pharmaceutical compounds are located only within the surface pores, i.e. not within the internal structure of the particle, which may be achieved by post-loading the particles with active pharmaceutical compounds after spray-drying a polymer solution. For example, the particles may be immersed in a solution or suspension of one or more active pharmaceutical compounds.

In some embodiments, the one or more active pharmaceutical compounds in the pharmaceutical composition are selected from one or more poorly soluble active pharmaceutical compounds as defined herein. The solubility of such compounds is increased by their loading onto the mesoporous particles produced during spray drying.

In some embodiments, the one or more active pharmaceutical compounds are selected from molecular species having a molecular weight of from 100 g/mol to 1000 g/mol, for example from 100 g/mol to 900 g/mol, from 100 g/mol to 800 g/mol, from 100 g/mol to 700 g/mol, from 100 g/mol to 600 g/mol, from 100 g/mol to 500 g/mol, from 150 g/mol to 450 g/mol, from 150 g/mol to 400 g/mol or from 200 g/mol to 400 g/mol.

Non-limiting examples of compounds which may be present in the composition include cardiovascular drugs such as Felodipine, Telmisartan, Valsartan, Carvedilol, Nifedipine, Nimodipine and Captopril; lipid-lowering drugs such as Lovastatin, Fenofibrate and Ezetimibe; antiviral drugs such as Atazanavir and Ritonavir; analgesics such as Ibuprofen, Meloxicam, Ketoprofen, Aceclofenac, Celecoxib, Indomethacin, Phenylbutazone and Flurbiprofen; anti-fungal drugs such as Itraconazole, Griseofulvin and Ketoconazole; antiepileptic drugs such as Carbamazepine, Oxcarbazepine and Rufinamide; anticancer drugs such as Camptothecin, Danazol and Paclitaxel; and other poorly soluble drugs such as Glibenclamide, Cyclosporine, Cinnarizine, Furosemide and Diazepam. One or a combination of two or more of these compounds may be present. In some embodiments, the one or more active pharmaceutical compounds are selected from one or more of Furosemide, Ibuprofen and Felodipine.

In some embodiments, the pharmaceutical composition consists of a particulate material according to the first aspect and one or more active pharmaceutical compounds. In other words, the composition may contain only a particulate material according to the first aspect and one or more active pharmaceutical compounds. This may ensure that no additional additives are present which may interfere with the amorphisation or activity of the active pharmaceutical compounds.

The pharmaceutical composition may be in powder form comprising a powder comprising a particulate material according to the first aspect. Such a powdered pharmaceutical composition offers a useful intermediate in the preparation of pharmaceutical dosage forms, for example tablets (which may be prepared by tabletting processes) or hard capsules (which may be prepared by capsule-filling processes).

The pharmaceutical composition may comprise the one or more active pharmaceutical compounds at a drug loading of from 1% w/w to 40% w/w, for example from 1% w/w to 30% w/w, from 2% w/w to 40% w/w, from 2% w/w to 35% w/w, from 2% w/w to 30% w/w, from 2% w/w to 29% w/w, from 2% w/w to 28% w/w, from 2% w/w to 27% w/w, from 2% w/w to 26% w/w, from 2% w/w to 25% w/w, from 5% w/w to 40% w/w, from 5% w/w to 35% w/w, from 5% w/w to 30% w/w, from 5% w/w to 25% w/w, from 10% w/w to 30% w/w, from 10% w/w to 25% w/w or from 15 wt % to 25 wt %. Herein, “% w/w” refers to the amount of compound with respect to the amount of particulate material alone. For example, a composition comprising 5 g of active pharmaceutical compound loaded onto 100 g of polymeric particles (to a total composition mass of 105 g) would have a drug loading of 5% w/w.

The particulate material of the invention is obtained or obtainable by spray-drying a polymer solution. In some embodiments, the particulate material of the invention is obtained by spray-drying a polymer solution.

Options and preferences for the polymer or polymers within the solution are as set out above in the context of the polymer or polymers making up the polymeric particles. So, for example, the polymer solution may comprise a cellulosic polymer.

The solution comprises a solvent and one or more polymers. The solvent may be a single solvent or a solvent mixture. In some embodiments, the solvent is a solvent mixture. In some embodiments, the solvent is a mixture of a polar protic solvent and a polar aprotic solvent. In some embodiments, the solvent is a mixture of water and an organic solvent, for example a polar organic solvent.

In some embodiments, the solvent is a mixture of a first solvent and a second solvent, wherein the first solvent is a solvent in which the one or more polymers is soluble and the second solvent is a solvent in which the one or more polymers is poorly soluble or insoluble, wherein “soluble” indicates that at least 1 g of the one or more polymers is soluble in 10 mL of solvent at 25° C., “poorly soluble” indicates that less than 1 g of the one or more polymers is soluble in 10 mL of solvent at 25° C. and “insoluble” indicates that very little or no amount of the one or more polymers is soluble in 10 mL of solvent at 25° C. It has been found that such a mixture of a first solvent in which the one or more polymers is soluble and a second solvent in which the one or more polymers is poorly soluble provides particularly good pore morphology in the spray-dried mesoporous polymeric particles, offering further improvement in the amorphisation and solubility of adsorbed compounds.

In some embodiments, the solvent mixture comprises at least 10% v/v of the first solvent and at least 5% v/v of the second solvent. In some embodiments, the solvent mixture comprises at least 20% v/v of the first solvent and at least 5% v/v of the second solvent. In some embodiments, the solvent mixture comprises at least 50% v/v of the first solvent and at least 5% v/v of the second solvent. In some embodiments, the solvent mixture comprises at least 60% v/v of the first solvent and at least 5% v/v of the second solvent. In some embodiments, the solvent mixture comprises at least 60% v/v of the first solvent and at least 5% v/v of the second solvent. In some embodiments, the solvent mixture comprises at least 80% v/v of the first solvent and at least 5% v/v of the second solvent.

In some embodiments, the solvent mixture comprises 75 to 95% v/v of the first solvent and 5 to 25% v/v of the second solvent, for example 80 to 90% v/v of the first solvent and 10 to 20% v/v of the second solvent.

In some embodiments, the solvent mixture consists of the first and second solvents. In some embodiments, the solvent mixture consists of at least 80% v/v of the first solvent and at least 10% v/v of the second solvent. In some embodiments, the solvent mixture consists of around 80% v/v of the first solvent and around 20% v/v of the second solvent. In some embodiments, the solvent mixture consists of 75 to 95% v/v of the first solvent and 5 to 25% v/v of the second solvent, for example 80 to 90% v/v of the first solvent and 10 to 20% v/v of the second solvent.

In some embodiments, the first solvent is acetone and the second solvent is water. In some embodiments, the first solvent is ethyl acetate and the second solvent is isopropanol.

In some embodiments, the solvent comprises a polar aprotic solvent (such as acetone) in an amount of greater than 50% v/v, for example at least 55% v/v, at least 60% v/v, at least 65% v/v, at least 70% v/v, at least 75% v/v or at least 80% v/v, with the balance being a polar protic solvent (such as water).

In some embodiments the solvent mixture comprises or consists of water and acetone. This particular mixture of solvents has been found to provide particularly good pore morphology in the spray-dried polymeric particles.

In some embodiments, the solvent mixture comprises or consists of acetone and water in a ratio of 80:20, 85:15 or 90:10 by volume.

The solution may be prepared by dissolving the one or more polymers in the solvent or solvent mixture. In some embodiments, the solution comprises at least 1% (w/v) polymer, for example at least 1.5% (w/v) polymer, at least 2% (w/v) polymer or at least 2.5% (w/v) polymer. In some embodiments, the balance of the solution is the solvent. In some embodiments, the solution comprises from 1% (w/v) to 20% (w/v) polymer, for example from 1.1% (w/v) to 18% (w/v) polymer, from 1.2% (w/v) to 15% (w/v) polymer, from 1.3% (w/v) to 12% (w/v) polymer, from 1.4% (w/v) to 10% (w/v) polymer, from 1.5% (w/v) to 10% (w/v) polymer, from 1.6% (w/v) to 8% (w/v) polymer, from 1.7% (w/v) to 6% (w/v) polymer, from 1.8% (w/v) to 5% (w/v) polymer or from 1.9% (w/v) to 3% (w/v) polymer. In some embodiments, the solution comprises around 2% (w/v) polymer. It will be understood that “% (w/v)” represents the weight of polymer in grams added to 100 mL of solvent. So, for example, when 4 g of polymer is added to 200 mL solvent to provide a solution, the solution contains 2% (w/v) polymer.

In some embodiments, the solution is free from any additives or templating agents. In some embodiments, the solution consists of the solvent and dissolved polymer. Templating agents (also called “pore-forming agents”) are traditionally used as a way to create porous materials. However in the present invention the porous polymeric particles form without the need for templating agents. This ensures that the final product is free of any contamination by templating agents which may affect the pharmaceutical acceptability of the product or interfere with the adsorption or solubility of drug compounds.

The polymer solution may be prepared by adding the one or more polymers to the solvent or solvent mixture and performing gentle mixing to effect dissolution and homogeneity. In some embodiments, the mixing is performed in a covered chamber to minimise solvent loss by evaporation. In some embodiments, mixing is performed with a magnetic stirred with a mixing speed of up to 500 rpm, for example up to 450 rpm, up to 400 rpm, up to 350 rpm, up to 300 rpm or up to 250 rpm. In some embodiments, mixing is performed at a temperature of from 10° C. to 30° C., for example from 12° C. to 28° C., from 15° C. to 25° C. or from 18° C. to 22° C. In some embodiments, mixing is performed for a period of from 15 to 120 mins, for example from 20 to 100 mins, from 25 to 90 mins or from 30 to 60 mins.

The polymer solution may be spray dried in the absence of any active pharmaceutical compounds to produce the mesoporous polymeric particles which are then subsequently contacted with one or more active pharmaceutical compounds to adsorb the compound onto the particle surface. However in other embodiments, in addition to the polymer, the polymer solution comprises one or more active pharmaceutical compounds. The solution containing both polymer and one or more active pharmaceutical compounds is then spray-dried to produce mesoporous particles pre-loaded with one or more active pharmaceutical compounds.

Such addition of one or more active pharmaceutical compounds to the polymer solution may be preferred when an extended or sustained release pharmaceutical composition is desired. The particles produced by such methods contain the active pharmaceutical compound not only adsorbed at the surface, but embedded within the particle, for example intimately dispersed within the particle polymer matrix or adsorbed to the surface of internal pores. The release of such active pharmaceutical compound from the particles in vivo is hindered, thereby providing an extended or sustained release composition in which active pharmaceutical compound is released more slowly over an extended period of time.

Without wishing to be bound by theory, it is believed that when polymer and active pharmaceutical compound are “co-spray dried” (i.e. both polymer and active pharmaceutical compound are present in the solution to be spray dried), the spray dried porous polymeric particles contain a greater amount of active pharmaceutical compound which is contained both within the internal structure of the particle and within pores at the surface of the particle. This is an alternative to “post-loading” techniques in which only the polymer is spray-dried and the spray dried particles are later contacted with active pharmaceutical compound to effect loading of the active pharmaceutical compound into the surface pores of the particle.

In some embodiments, the amount of the one or more active pharmaceutical compounds present in the polymer solution (i.e. the drug loading of the polymer solution) is from 1% w/w to 40% w/w, for example from 1% w/w to 30% w/w, from 2% w/w to 40% w/w, from 2% w/w to 35% w/w, from 2% w/w to 30% w/w, from 2% w/w to 29% w/w, from 2% w/w to 28% w/w, from 2% w/w to 27% w/w, from 2% w/w to 26% w/w, from 2% w/w to 25% w/w, from 5% w/w to 40% w/w, from 5% w/w to 35% w/w, from 5% w/w to 30% w/w, from 5% w/w to 25% w/w, from 10% w/w to 30% w/w, from 10% w/w to 25% w/w or from 15 wt % to 25 wt %, wherein “% w/w” refers to the amount of one or more active pharmaceutical compounds with respect to the amount of polymer alone. For example, a polymer solution comprising 5 g of active pharmaceutical compound and 100 g of polymer (to a total mass of 105 g) would have a drug loading of 5% w/w.

In some embodiments, the one or more active pharmaceutical compounds in the solution are selected from one or more poorly soluble active pharmaceutical compounds. The solubility of such compounds is increased by their loading onto the mesoporous particles produced during spray drying.

In some embodiments, the one or more active pharmaceutical compounds are selected from molecular species having a molecular weight of from 100 g/mol to 1000 g/mol, for example from 100 g/mol to 900 g/mol, from 100 g/mol to 800 g/mol, from 100 g/mol to 700 g/mol, from 100 g/mol to 600 g/mol, from 100 g/mol to 500 g/mol, from 150 g/mol to 450 g/mol, from 150 g/mol to 400 g/mol or from 200 g/mol to 400 g/mol.

Non-limiting examples of compounds which may be added to the solution include cardiovascular drugs such as Felodipine, Telmisartan, Valsartan, Carvedilol, Nifedipine, Nimodipine and Captopril; lipid-lowering drugs such as Lovastatin, Fenofibrate and Ezetimibe; antiviral drugs such as Atazanavir and Ritonavir; analgesics such as Ibuprofen, Meloxicam, Ketoprofen, Aceclofenac, Celecoxib, Indomethacin, Phenylbutazone and Flurbiprofen; anti-fungal drugs such as Itraconazole, Griseofulvin and Ketoconazole; antiepileptic drugs such as Carbamazepine, Oxcarbazepine and Rufinamide; anticancer drugs such as Camptothecin, Danazol and Paclitaxel; and other poorly soluble drugs such as Glibenclamide, Cyclosporine, Cinnarizine, Furosemide and Diazepam. One or a combination of two or more of these compounds may be dissolved in the polymer solution. In some embodiments, the one or more active pharmaceutical compounds are selected from one or more of Furosemide, Ibuprofen and Felodipine.

Another aspect of the invention is a method of manufacturing a particulate material comprising spray-drying a polymer solution, the particulate material comprising porous polymeric particles, the average pore diameter being from 2 to 50 nm, wherein the porous polymeric particles have a volume mean diameter D[4,3] of less than 100 μm.

In the method of manufacturing a particulate material, the polymer, polymer solution and the particulate material itself are as discussed above in the context of the first aspect.

In some embodiments, the method comprises a preliminary step of preparing a polymer solution, comprising dissolving one or more polymers in a solvent. The solvent and one or more polymers may be as described above in relation to the first aspect. For example, the solvent may be an acetone:water mixture and the polymer may be a cellulosic polymer. The preliminary step may also include dissolving one or more active pharmaceutical compounds in the solvent along with the polymer. In other embodiments the preparation of the polymer solution comprises the mixing of only the polymer and the solvent, i.e. the solution contains only polymer and solvent and no further additives or excipients.

To form the porous polymeric particles, the above-described polymer solution is subjected to a spray-drying process. Such processes are well-known to the skilled person.

Any suitable spray-drying apparatus may be used in the method of the invention.

In some embodiments, the inlet temperature is from 60 to 175° C., for example from 60 to 170° C., 60 to 165° C., 60 to 160° C., 60 to 155° C., 60 to 150° C., 60 to 145° C. or 60 to 140° C. In some embodiments, the inlet temperature is about 100° C.

In some embodiments, the inlet temperature during spray-drying of the polymer solution is lower than the glass transition temperature T_{g} of the polymer in the polymer solution. So, for example, if the glass transition temperature of the polymer is 130° C., the inlet temperature during spray-drying may be less than 130° C. In this way, pore formation is facilitated and the formation of mesopores of the correct size is promoted.

The spray-dryer may be operated in closed mode. The spray-dryer may utilise an inert carrier gas, for example nitrogen or carbon dioxide. An atomisation pressure of from 100 to 500 kPa may be used during spray-drying, for example from 100 to 450 kPa, 100 to 400 kPa, 100 to 350 kPa, 100 to 300 kPa, 150 to 250 kPa or about 200 kPa.

In some embodiments, the spray-drying is carried out in a spray-dryer under closed-mode with nitrogen, an inlet temperature of from 60 to 180° C. and an atomisation pressure of from 100 to 500 kPa.

Suitable spray-drying apparatus which may be used in the present invention includes the mini spray dryer Buchi B-290 with the Inert Loop Buchi B-295 (Flawil, Switzerland).

The particular flow rates used during spray drying will depend on the choice of spray dryer and the scale of manufacture. For the above-mentioned mini spray dryer Buchi B-290 with the Inert Loop Buchi B-295, a feed flow rate (flow rate of the polymer solution) of from 1 mL/min to 10 mL/min may be used during the spray drying process, for example from 2 mL/min to 8 mL/min, from 3 mL/min to 6 mL/min or about 5 mL/min. An inert gas flow rate of from 200 L/hour to 1000 L/hour may be used during the spray drying process, for example from 250 L/hour to 1000 L/hour, 400 L/hour to 800 L/hour or about 600 L/hour. In some embodiments, the inert gas is nitrogen. A drying gas flow rate of from 10 m³/hour to 50 m³/hour may be used during the spray drying process, for example from 15 m³/hour to 45 m³/hour, 20 m³/hour to 40 m³/hour, 24 m³/hour to 35 m³/hour or about 30 m³/hour.

The choice of spray drying apparatus is not particularly limited and the spray dryer may be chosen based on e.g. the scale of manufacture required. For pilot scale manufacture a larger spray dryer may be employed, for example the Niro Mobile Minor spray dryer. The skilled person will understand that the above mentioned feed flow rate, inert gas (atomisation) flow rate and drying gas flow rate will change accordingly based on the size of the spray dryer and the skilled person is able to choose suitable flow rates.

For example, for the Niro Mobile Minor spray dryer the feed flow rate (flow rate of the polymer solution) may be from 1.0 kg/hour to 6.0 kg/hour, the inert gas (atomisation) flow rate may be from 4 kg/hour to 25 kg/hour and the drying gas flow rate may be from 10 kg/hour to 80 kg/hour.

The outlet temperature during spray drying is a function of various process parameters such as inlet temperature, feed rate and flow rate, but generally may be within the range 40 to 120° C.

In some embodiments, the method comprises one or more processing steps performed on the particulate material after spray-drying. For example, the material may be subjected to one or more drying steps to remove any residual solvent.

In some embodiments, the method comprises a step of contacting the spray-dried particulate material with one or more active pharmaceutical compounds. In some embodiments, the method comprises a step of contacting the spray-dried particulate material with a solution of one or more active pharmaceutical compounds (“drug solution”). This may be achieved by dissolving the one or more active pharmaceutical compounds in a suitable solvent and combining the solution with the particulate material to create a suspension. In this way, the active pharmaceutical compound becomes loaded onto the surface of the particles, i.e. adsorbed onto the surface, including within the mesopores. The suspension may be stirred to improve loading efficiency. In some embodiments, the solvent in which the one or more active pharmaceutical compounds are dissolved is an alcohol. In some embodiments, the solvent is ethanol.

In some embodiments, the amount of the one or more active pharmaceutical compounds in the drug solution is at least 2 mg/mL, for example at least 2.5 mg/mL, at least 3 mg/mL, at least 3.5 mg/mL, at least 4 mg/mL, at least 4.5 mg/mL or at least 5 mg/mL. In some embodiments, the amount of the one or more active pharmaceutical compounds in the drug solution is up to 50 mg/mL, for example up to 45 mg/mL, up to 40 mg/mL, up to 35 mg/mL, up to 30 mg/mL, up to 25 mg/mL or up to 20 mg/mL. In some embodiments, the amount of the one or more active pharmaceutical compounds in the drug solution is from 2 to 50 mg/mL, for example from 2 to 40 mg/mL, from 2 to 30 mg/mL, from 5 to 20 mg/mL, from 5 to 15 mg/mL or about 10 mg/mL.

In some embodiments, the drug loading in the drug solution is from 1% w/w to 40% w/w, for example from 1% w/w to 30% w/w, from 2% w/w to 40% w/w, from 2% w/w to 35% w/w, from 2% w/w to 30% w/w, from 2% w/w to 29% w/w, from 2% w/w to 28% w/w, from 2% w/w to 27% w/w, from 2% w/w to 26% w/w, from 2% w/w to 25% w/w, from 5% w/w to 40% w/w, from 5% w/w to 35% w/w, from 5% w/w to 30% w/w, from 5% w/w to 25% w/w, from 10% w/w to 30% w/w, from 10% w/w to 25% w/w or from 15 wt % to 25 wt %, wherein “% w/w” refers to the amount of one or more active pharmaceutical compounds with respect to the amount of particulate material alone added to the drug solution. For example, a drug solution comprising 5 g of active pharmaceutical compound and 100 g of polymeric particles (to a total mass of 105 g) would have a drug loading of 5% w/w.

In some embodiments the solution contains one active pharmaceutical compound.

In some embodiments the suspension of the particles in the drug solution is agitated, or stirred. This promotes the uptake of active pharmaceutical compound by the particles in the suspension.

The suspension may be left for a period of at least an hour, for example at least 2 hours, at least 5 hours or at least 10 hours, optionally with stirring. The suspension may be left for a period of up to 20 hours, for example up to 18 hours, up to 15 hours or up to 12 hours, optionally with stirring.

After the suspension has been left for a suitable amount of time to provide the desired drug loading, the drug-loaded particulate material may be separated from the suspension, for example by filtration or spray-drying. In some embodiments, the suspension of porous particles in the drug solution is spray-dried. The conditions for spray-drying may be as set out above in the context of the spray-drying of the polymer solution. In some embodiments, after filtration or spray drying, the material is subjected to a further drying step, for example in an oven or other high ambient temperature environment.

In some embodiments, drying of the drug-loaded particulate material is carried out until the residual solvent content of the material is less than or equal to 0.5 wt % based on the total weight of particulate material, solvent and active pharmaceutical compound, for example less than or equal to 0.4 wt %, less than or equal to 0.3 wt % or less than or equal to 0.2 wt %. This can be achieved for example by providing a longer residence time in the spray-dryer, or by performing an additional drying step for a sufficient period of time.

Alternative methods of loading active pharmaceutical onto the particulate material may be used, for example solvent-free methods. These have the advantage that no subsequent drying step to remove solvent is required. However, in general solvent-based methods are preferred because a higher drug loading efficiency is possible.

As explained above, the porous particulate material of the invention may be loaded with one or more active pharmaceutical compounds. In some embodiments, the surface of the porous particulate material is loaded with one or more active pharmaceutical compounds. In some embodiments, the porous particulate material is loaded with one active pharmaceutical compound (i.e. a single type/species of compound).

The active pharmaceutical compound or compounds which may be loaded onto the material of the invention is not particularly limited. It may be particularly useful to load one or more compounds of poor solubility onto the material, since adsorption into the mesopores of the material may increase the solubility, thereby improving the usefulness of the compound.

In some embodiments, the one or more active pharmaceutical compounds are each independently selected from compounds in BCS Class II or BCS Class IV according to the US Food and Drug Administration's guidance. In some embodiments, the one or more active pharmaceutical compounds are each independently selected from compounds which are sparingly soluble, slightly soluble, very slightly soluble or practically insoluble as defined in Solubility—Part III— General Notices of British Pharmacopoeia (BP) 2019 and European Pharmacopoeia (EP) 9th Edition.

In some embodiments, the one or more active pharmaceutical compounds are selected from molecular species having a molecular weight of from 100 g/mol to 1000 g/mol, for example from 100 g/mol to 900 g/mol, from 100 g/mol to 800 g/mol, from 100 g/mol to 700 g/mol, from 100 g/mol to 600 g/mol, from 100 g/mol to 500 g/mol, from 150 g/mol to 450 g/mol, from 150 g/mol to 400 g/mol or from 200 g/mol to 400 g/mol.

In some embodiments, the one or more active pharmaceutical compounds are selected from compounds having a log P of not greater than 5, for example not greater than 4.5, not greater than 4, not greater than 3.5 or not greater than 3, wherein P is the octanol-water partition coefficient determined at 25° C. (also denoted “Pow”).

As is well-understood by the skilled person, the partition coefficient P is the ratio of concentrations of a compound between two specified solvents (in this case, octanol and water), and log P is the logarithm of that ratio. Log P is therefore a measure of lipophilicity or hydrophobicity. A higher value of log P indicates a more lipophilic compound.

In some embodiments, the one or more active pharmaceutical compounds are selected from compounds having a molecular weight of not greater than 500 g/mol and a log P of not greater than 5, wherein P is the octanol-water partition coefficient determined at 25° C.

In some embodiments, the one or more active pharmaceutical compounds are selected from compounds which are sparingly soluble, slightly soluble, very slightly soluble or practically insoluble as defined in Solubility—Part III—General Notices of British Pharmacopoeia (BP) 2019 and European Pharmacopoeia (EP) 9th Edition and have one or more of a log P of not greater than 5, wherein P is the octanol-water partition coefficient determined at 25° C., and a molecular weight of not greater than 500 g/mol.

In some embodiments, the one or more active pharmaceutical compounds are selected from compounds which are sparingly soluble, slightly soluble, very slightly soluble or practically insoluble as defined in Solubility—Part III—General Notices of British Pharmacopoeia (BP) 2019 and European Pharmacopoeia (EP) 9th Edition and have a log P of not greater than 5, wherein P is the octanol-water partition coefficient determined at 25° C., and a molecular weight of not greater than 500 g/mol.

Non-limiting examples of compounds which may be loaded onto the material of the invention include cardiovascular drugs such as Felodipine, Telmisartan, Valsartan, Carvedilol, Nifedipine, Nimodipine and Captopril; lipid-lowering drugs such as Lovastatin, Fenofibrate and Ezetimibe; antiviral drugs such as Atazanavir and Ritonavir; analgesics such as Ibuprofen, Meloxicam, Ketoprofen, Aceclofenac, Celecoxib, Indomethacin, Phenylbutazone and Flurbiprofen; anti-fungal drugs such as Itraconazole, Griseofulvin and Ketoconazole; antiepileptic drugs such as Carbamazepine, Oxcarbazepine and Rufinamide; anticancer drugs such as Camptothecin, Danazol and Paclitaxel; and other poorly soluble drugs such as Glibenclamide, Cyclosporine, Cinnarizine, Furosemide and Diazepam. One or a combination of two or more of these compounds may be loaded onto the particulate material of the invention to improve solubility and/or provide an extended or sustained release profile. In some embodiments, the one or more active pharmaceutical compounds are selected from one or more of Furosemide, Ibuprofen and Felodipine.

Thus another aspect of the invention is a pharmaceutical composition comprising a particulate material according to the first aspect loaded with one or more active pharmaceutical compounds. In some embodiments, the particulate material is surface-loaded with one or more active pharmaceutical compounds. In some embodiments, the one or more active pharmaceutical compounds are selected from one or more of the compounds listed above.

In some embodiments, the pharmaceutical composition is an enhanced-solubility Felodipine composition comprising a particulate material according to the first aspect and Felodipine adsorbed onto the surface of the particulate material. Some aspects of the invention provide the enhanced-solubility Felodipine composition for use in therapy. Some aspects of the invention provide the enhanced-solubility Felodipine composition for use in the treatment of a disease or disorder selected from high blood pressure and stable angina. Some aspects of the invention provide methods of treating a patient suffering from a disease or disorder selected from high blood pressure and stable angina, comprising administering to the patient a therapeutically acceptable amount of the enhanced-solubility Felodipine composition described above.

In some embodiments, the pharmaceutical composition is an enhanced-solubility Furosemide composition comprising a particulate material according to the first aspect and Furosemide adsorbed onto the surface of the particulate material. Some aspects of the invention provide the enhanced-solubility Furosemide composition for use in therapy. Some aspects of the invention provide the enhanced-solubility Furosemide composition for use in the treatment of a disease or disorder selected from oedema and hypertension. Some aspects of the invention provide methods of treating a patient suffering from a disease or disorder selected from oedema and hypertension, comprising administering to the patient a therapeutically acceptable amount of the enhanced-solubility Furosemide composition described above.

In some embodiments, the pharmaceutical composition is an enhanced-solubility Ibuprofen composition comprising a particulate material according to the first aspect and Ibuprofen adsorbed onto the surface of the particulate material. Some aspects of the invention provide the enhanced-solubility Ibuprofen composition for use in therapy. Some aspects of the invention provide the enhanced-solubility Ibuprofen composition for use in the treatment of a disease or disorder selected from pain, fever and inflammation. Some aspects of the invention provide methods of treating a patient suffering from a disease or disorder selected from pain, fever and inflammation, comprising administering to the patient a therapeutically acceptable amount of the enhanced-solubility Ibuprofen composition described above.

An aspect of the invention is a dosage form comprising the pharmaceutical composition of the second aspect. In some embodiments, the dosage form is an oral dosage form. In some embodiments, the dosage form is a tablet or capsule.

The dosage form may additionally comprise one or more pharmaceutically acceptable binders, carriers, diluents or excipients well-known to the skilled person.

Some aspects of the invention provide a pharmaceutical composition as described above, for use in therapy. Some aspects of the invention provide the use of a pharmaceutical composition as described above in the manufacture of a medicament. Some aspects of the invention provide a method of treatment of the human or animal body, comprising administration of a therapeutically effective amount of a pharmaceutical composition described above to a patient in need thereof. Other aspects of the invention provide a method of treating the human or animal body, comprising administering to a patient in need thereof a therapeutically effective amount of a pharmaceutical composition described above.

A wide range of diseases or disorders may be treated in these aspects, depending on the particular active pharmaceutical compound or compounds which are loaded onto the particulate material.

An aspect of the invention is a method of improving the solubility of an active pharmaceutical compound, comprising loading the compound onto the particulate material according to the first aspect. The active pharmaceutical compound may be one of the compounds mentioned above.

An aspect of the invention is the use of a particulate material according to the first aspect as a solubility-enhancing carrier for one or more active pharmaceutical compounds.

In some embodiments, apparent solubility is increased by a factor of at least 1.1, for example at least 1.15, at least 1.2, at least 1.25 or at least 1.3 through this method. In some cases, solubility is increased by a factor of up to around 10.

The present invention also relates to a means to provide an extended release (or sustained release) composition of an active pharmaceutical compound. When the polymer solution also contains an active pharmaceutical compound, the compound becomes at least partially entrapped within the porous polymeric particles after spray-drying. The release of the compound from the particles is thereby limited and becomes extended, or sustained, over a longer period of time.

Thus the invention also provides a method of manufacturing an extended release pharmaceutical composition, comprising spray-drying a solution comprising polymer and one or more active pharmaceutical compounds to form a particulate material, the particulate material comprising porous polymeric particles, the average pore diameter being from 2 to 50 nm, wherein the porous polymeric particles have a volume mean diameter D[4,3] of less than 100 μm.

### SUMMARY OF THE FIGURES

So that the invention may be understood, and so that further aspects and features thereof may be appreciated, embodiments illustrating the principles of the invention will now be discussed in further detail with reference to the accompanying figures, in which:

FIG. 1 shows SEM images of mesoporous cellulose acetate butyrate particles according to the invention, prepared by a spray drying process, including (a) a CAB particle cross-section at a magnification of ×5000 and a scale bar of 1 μm and (b) the internal mesoporous structure of a particle at a magnification of ×30,000 and a scale bar of 100 nm.

FIG. 2 shows SEM images of mesoporous cellulose acetate butyrate particles according to the invention, prepared by a spray drying process, including (a) a CAB particle surface at a magnification of ×5000 and a scale bar of 1 μm, and (b) a CAB particle surface at a magnification of ×33,000 and a scale bar of 100 nm.

FIG. 3 shows DSC thermograms of Felodipine raw material (solid line) and Felodipine-loaded mesoporous CAB particles (dashed line), under a scanning rate of 10° C./min and a scanning range of 50-250° C.

FIG. 4 shows DSC thermograms of Ibuprofen raw material (solid line) and Ibuprofen-loaded mesoporous CAB particles (dashed line), under a scanning rate of 10° C./min and a scanning range of 40-250° C.

FIG. 5 shows DSC thermograms of Furosemide raw material (solid line) and Furosemide-loaded mesoporous CAB particles (dashed line), under a scanning rate of 10° C./min and a scanning range of 100-300° C.

FIG. 6 shows dissolution profiles of Felodipine raw material (solid line) and Felodipine-loaded mesoporous CAB particles (dashed line). Testing conditions: phosphate buffer pH 6.5+0.25% SLS, 500 mL, USP apparatus 1 (rotating basket), 50 rpm, HPLC method (mobile phase: pH 3 phosphate buffer:acetonitrile:methanol (30:45:25); column: C18, 15 cm×4.6 mm, 5 μm; flow rate: 1 mL/min; injection volume: 40 μL; detector: UV, 362 nm).

FIG. 7 shows dissolution profiles of Ibuprofen raw material (solid line) and Ibuprofen-loaded mesoporous CAB particles (dashed line). Testing conditions: HCL-NaCl medium pH 3+0.25% SLS, 900 mL, USP apparatus 1 (rotating basket), 100 rpm, HPLC method (mobile phase: pH 3 phosphate buffer:acetonitrile (60:40); column: C18, 15 cm×4.6 mm, 5 μm; flow rate: 2 mL/min; injection volume: 20 μL; detector: UV, 254 nm).

FIG. 8 shows dissolution profiles of Furosemide raw material (solid line) and Furosemide-loaded mesoporous CAB particles (dashed line). Testing conditions: HCL-NaCl medium pH 3+0.25% SLS, 900 mL, USP apparatus 1 (rotating basket), 100 rpm, HPLC method (mobile phase: pH 3 phosphate buffer:acetonitrile (60:40); column: C18, 15 cm×4.6 mm, 5 μm; flow rate: 1 mL/min; injection volume: 10 μL; detector: UV, 234 nm).

FIG. 9 shows dissolution profiles of Felodipine raw material (dotted line with triangular markers), spray-dried raw Felodipine (dotted line with square markers) and Felodipine-loaded mesoporous CAB particles prepared by co-spray drying a solution containing CAB and three different levels of Felodipine: 5 wt %, 15 wt % and 25 wt % (solid lines). Testing conditions: phosphate buffer pH 6.5+0.25% SLS, 500 mL, USP apparatus 1 (rotating basket), 50 rpm, HPLC method (mobile phase: pH 3 phosphate buffer:acetonitrile:methanol (30:45:25); column: C18, 15 cm×4.6 mm, 5 μm; flow rate: 1 mL/min; injection volume: 40 μL; detector: UV, 362 nm).

FIG. 10 shows SEM images of mesoporous cellulose acetate butyrate particles according to the invention, prepared by co-spray drying a solution of CAB and Felodipine, including (a) drug loading of 5 wt %, (b) drug loading of 10 wt % and (c) drug loading of 25 wt %. SEM images were taken at 30,000× magnification with a scale bar of 100 nm.

FIG. 11 shows CLSM images of mesoporous cellulose acetate butyrate particles loaded with fluorescein by two different methods (a) post-loading with fluorescein, and (b) co-spray drying with fluorescein.

FIG. 12 shows plots of (a) cumulative distribution of pore volume of particles of Sample 8, and (b) the pore size distribution curve for the particles of Sample 8 determined according to the BJH method.

### EXAMPLES

Aspects and embodiments of the present invention will now be discussed in the following examples. Further aspects and embodiments will be apparent to those skilled in the art. All documents mentioned in this text are incorporated herein by reference.

### Characterisation of Particle Properties

In the Examples below, the pore size, pore volume and specific surface area of polymeric mesoporous particles were analysed by gas adsorption porosimetry using pore size analyser Quantachrome Nova 4200e. Each sample was degassed under vacuum at 100° C. for 24 h before obtaining nitrogen adsorption-desorption measurements.

Morphology of mesoporous particles was examined by scanning electron microscopy (SEM) in JEOL JSM-7800F operating at 1 kV under a high vacuum. The samples were not gold-coated to retain sample integrity, i.e. original surface features. Approximately 1 mg of each sample was placed onto a double-sided adhesive strip on a sample holder.

Particle size of samples was determined by laser diffraction using particle size analyser Sympatec HELOS/BR and dry disperser RODOS with feeder VIBRI. The measuring range was 0-195 μm. Approximately 0.2 g of each sample was placed in the feeder tray. The time of each measurement was 10 s with powder dispensing pressure of 300 kPa. The results were obtained as volume mean diameter (VMD; D[4,3]) and given as the average of three analyses for each sample.

To assess the level of drug loading of the particles, a known amount of drug-loaded mesoporous particles were dissolved in 25 ml of acetone and diluted with a corresponding dissolution medium to 500 ml, then sonicated for 30 min.

The concentrations of dissolved drug were then determined using HPLC in a C18 column (15 cm×4.6 mm, 5 μm) and UV detector at 362 nm in an Agilent 1200 HPLC system.

### Example 1—Preparation of Cellulosic Mesoporous Particles

4 g of cellulose acetate butyrate (CAB) or cellulose acetate (CA), or ethyl cellulose (EC) were dissolved in 200 mL of either the acetone:water or ethyl acete:isopropanol mixtures prepared at a volume ratio of 90:10. The resulting polymer solutions were then spray dried with a two-fluid nozzle. A mini spray dryer Buchi B-290 in closed mode with nitrogen in the Inert Loop Buchi B-295 (Flawil, Switzerland) was used with a feed rate of 5 mL/min, nitrogen flow rate of 600 L/h, atomization pressure of 200 KPa, and a drying gas flow rate of 30 m³/h. The spray drying process was operated with an inlet temperature of 100° C. All materials and solvent were pharmaceutical grade.

Table 1 below shows the results of measurements taken on the particulate material produced in the spray drying.

TABLE 1

| | | | | Average | | |
|---|---|---|---|---|---|---|
| | | | Surface | pore | Pore | Particle |
| | | | area | diameter | volume | size |
| Polymer | Sample # | Solvent mixture | (cm²/g) | (nm) | (cm³/g) | (μm) |
| Mesoporous | 1 | Acetone:water | 12.8 | 16.3 | 0.05 | 48.8 |
| ethyl | | | | | | |
| cellulose (EC) | | | | | | |
| Mesoporous | 2 | Acetone:water | 32.6 | 24.9 | 0.18 | 22.5 |
| cellulose | | | | | | |
| acetate | | | | | | |
| butyrate (CAB) | | | | | | |
| Mesoporous | 3 | Acetone:water | 15.7 | 24.8 | 0.09 | 32.6 |
| cellulose | | | | | | |
| acetate (CA) | | | | | | |
| Mesoporous | 4 | Ethyl | 6.6 | 15.1 | 0.02 | 19.3 |
| cellulose | | acetate:isopropanol | | | | |
| acetate | | | | | | |
| butyrate (CAB) | | | | | | |

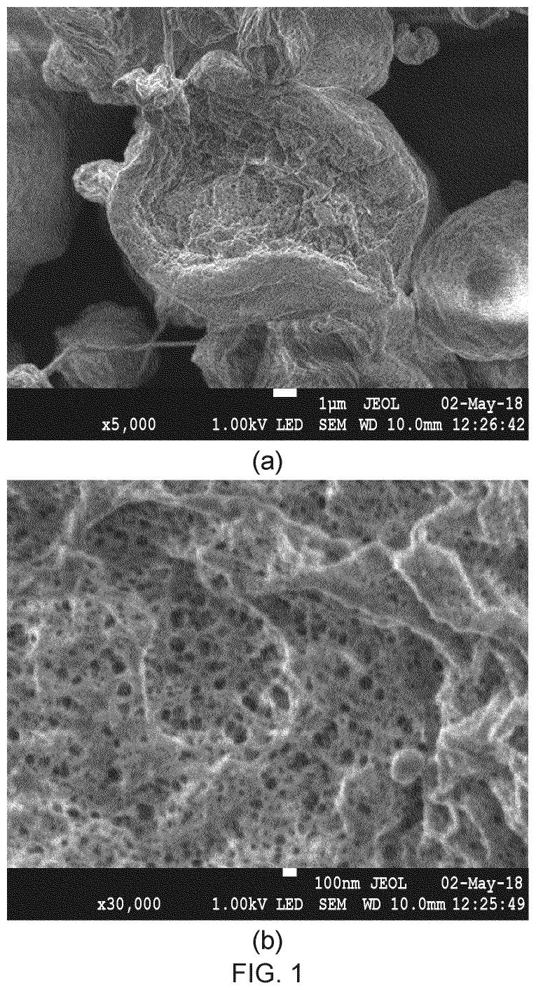

FIG. 1 shows SEM images of the particles of Sample 2. FIG. 1(a) is a cross-section of a broken particle showing the porous internal structure at a magnification of ×5000. FIG. 1(b) shows the same particle cross-section at a magnification of ×30,000, which shows the mesoporous internal structure in greater detail.

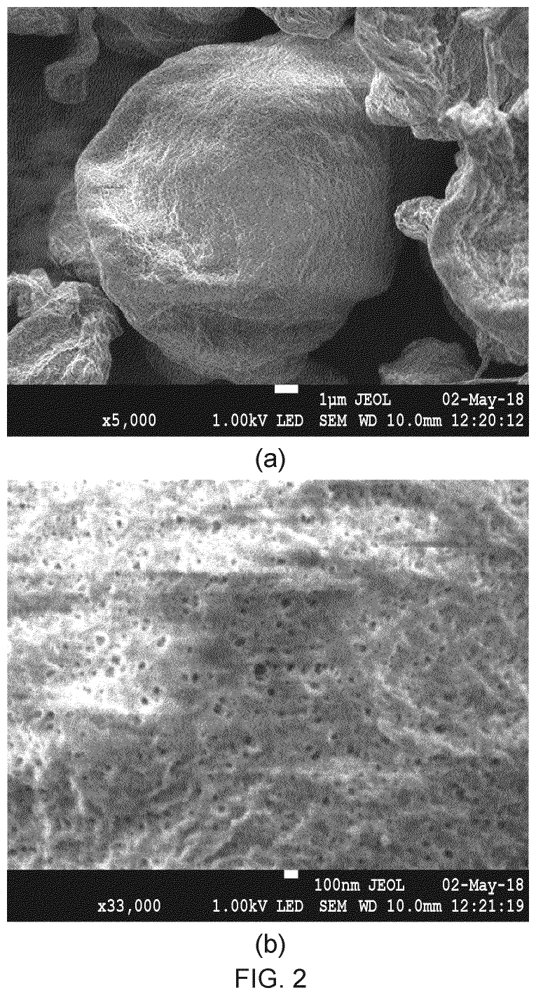

FIG. 2 shows SEM images of the particles of Sample 2. FIG. 2(a) is the external surface of a particle showing the porous surface structure at a magnification of ×5000. FIG. 2(b) shows the same particle surface at a magnification of ×33,000, which shows the mesoporous surface structure in greater detail.

### Example 2—Preparation of CAB Polymeric Mesoporous Particles

Polymeric mesoporous particles were manufactured from various types of CAB having a butyryl content ranging from about 15% to about 60%, an acetyl content ranging from about 1% to about 30%, and a hydroxyl content ranging from about 0.5% to about 5% (ex Eastman Chemical). Solvent mixtures of acetone:water were prepared at volume ratios of 80:20, 85:15 and 90:10. 4 g of CAB were dissolved in 200 mL of solvent mixtures. Polymer solutions were then spray dried with a two-fluid nozzle. A mini spray dryer Buchi B-290 in closed mode with nitrogen in the Inert Loop Buchi B-295 (Flawil, Switzerland) was used with a feed rate of 5 mL/min, nitrogen flow rate of 600 L/h, atomization pressure of 200 kPa, and drying gas flow rate of 30 m³/h. The spray drying process was operated with inlet temperature in the range of 60-140° C. All materials and solvents were pharmaceutical grade.

Table 2 below shows details of the various samples produced:

TABLE 2

| | | | | Average | |
|---|---|---|---|---|---|
| | Inlet | Solvent ratio | Surface | pore | Pore |
| | temp. | (v/v) | area | diameter | volume |
| Sample | (° C.) | (acetone:water) | (cm²/g) | (nm) | (cm³/g) |
| 5 | 60 | 80:20 | 45.6 ± 2.1 | 24.9 ± 0.6 | 0.29 ± 0.01 |
| 6 | 100 | 80:20 | 42.5 ± 0.6 | 22.6 ± 2.2 | 0.24 ± 0.04 |
| 7 | 140 | 80:20 | 42.8 ± 3.3 | 20.4 ± 0.7 | 0.22 ± 0.01 |
| 8 | 60 | 85:15 | 56.7 ± 6.9 | 20.8 ± 2.3 | 0.32 ± 0.03 |
| 9 | 100 | 85:15 | 44.3 ± 1.3 | 22.0 ± 1.3 | 0.25 ± 0.02 |
| 10 | 140 | 85:15 | 38.6 ± 5.9 | 23.9 ± 0.6 | 0.23 ± 0.03 |
| 11 | 60 | 90:10 | 42.5 ± 2.9 | 27.2 ± 0.1 | 0.29 ± 0.02 |
| 12 | 100 | 90:10 | 32.6 ± 1.0 | 24.1 ± 1.2 | 0.18 ± 0.01 |
| 13 | 140 | 90:10 | 19.0 ± 3.6 | 27.0 ± 1.3 | 0.13 ± 0.01 |

### Example 3—Preparation of Felodipine-Loaded Mesoporous Particles

Mesoporous CAB particles of Sample 8 in Table 2 were added to a solution of Felodipine (FELO, complies with USP 36, purity >98%) in ethanol (10 mg/mL) to form a suspension at an initial drug load of 15% (w/w). The suspension was gently stirred for 12 h, then spray-dried at inlet temperature of 100° C. using a mini spray dryer Buchi B-290 and inert loop Buchi B-295 in closed mode with nitrogen flow rate of 600 L/min, feed rate of 5 mL/min, and drying gas flow rate of 30 m³/h. All materials and solvent were pharmaceutical grade.

### Example 4—Preparation of Ibuprofen-Loaded Mesoporous Particles

Mesoporous CAB particles of Sample 8 in Table 2 were added to a solution of Ibuprofen (IBU, purity >98%) in ethanol (10 mg/mL) to form a suspension at an initial drug load of 20% (w/w). The suspension was gently stirred for 12 h, then spray-dried at inlet temperature of 80° C. using a mini spray dryer Buchi B-290 and inert loop Buchi B-295 under the same process parameters as Example 3. All materials and solvent were pharmaceutical grade.

### Example 5—Preparation of Furosemide-Loaded Mesoporous Particles

Mesoporous CAB particles of Sample 8 in Table 2 were added to a solution of Furosemide (FURO, complies with USP 38, purity >99%) in ethanol (10 mg/mL) to form a suspension at an initial drug load of 21% (w/w). The suspension was gently stirred for 12 h, then spray-dried using the same apparatus and under the same process parameters as Example 3. All materials and solvent were pharmaceutical grade.

### Example 6—Co-Spray Dried Felodipine-CAB Polymeric Particles for Extended Release

4.0 g of CAB was mixed with 0.2 g, 0.6 g and 1.0 g of Felodipine to produce mixtures of polymer and drug with 5, 15 and 25% drug loading (w/w), respectively (i.e. drug loading in % w/w herein is calculated by dividing the mass of drug compound added to the solution by the mass of polymeric particles added to the solution, then multiplying by 100). These mixtures were then each dissolved in 200 mL of acetone:water at ratio of 85:15 (v/v) and co-spray dried using a mini spray dryer Buchi B-290 in closed mode with nitrogen in the Inert Loop Buchi B-295 (Flawil, Switzerland), inlet temperature of 100° C., nitrogen flow rate of 600 L/min, feed rate of 5 mL/min, and drying gas flow rate of 30 m³/h. All materials and solvent were pharmaceutical grade.

Table 3 below sets out the properties of the co-spray dried Felodipine-CAB porous particles (n=3; mean±standard deviation).

TABLE 3

| | | | Average | |
|---|---|---|---|---|
| | Felodipine | Surface | pore | |
| Sample | loading | area | diameter | Pore volume |
| # | (% w/w) | (cm²/g) | (nm) | (cm³/g) |
| 14 | 5 | 46.3 ± 2.5 | 23.7 ± 0.6 | 0.28 ± 0.02 |
| 15 | 15 | 14.2 ± 3.4 | 17.2 ± 2.5 | 0.06 ± 0.01 |
| 16 | 25 | 10.4 ± 0.4 | 13.7 ± 1.4 | 0.04 ± 0.01 |

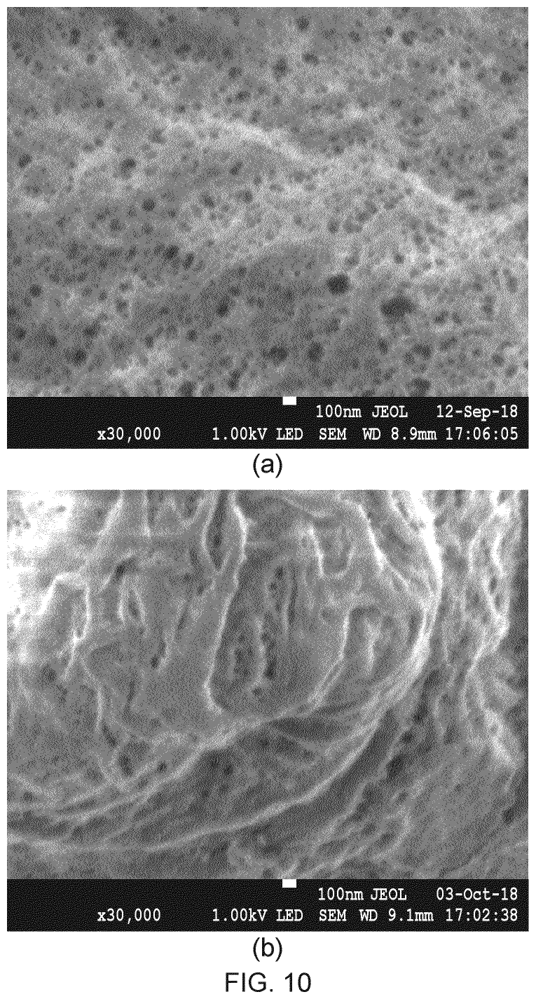

SEM images of the particles having different levels of drug loading are shown in FIG. 10.

### Example 7—Thermal Analysis of Drug-Loaded Mesoporous Particles

Thermal properties of the drug-loaded mesoporous particles made in Examples 3-5 were characterised by DSC instrument TA Q 200. Samples were accurately weighed (approximately 3-5 mg) into Tzero aluminium pans and heated in the temperature range of 50-300° C. at a scanning rate of 10° C./min under nitrogen. TA universal analysis 2000 software (version 4.5) was employed to analyse the resulting DSC graphs.

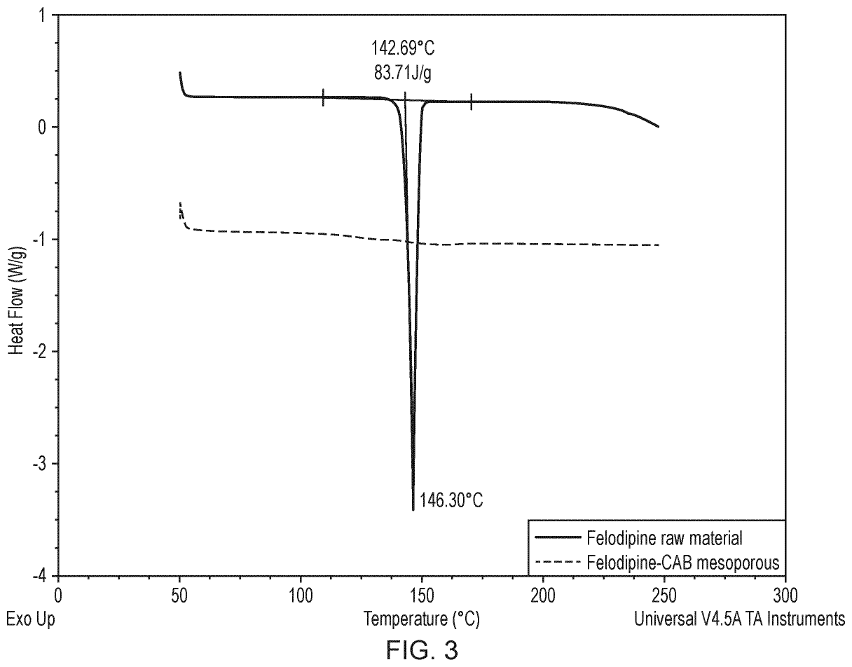

FIGS. 3-5 show the DSC thermograms for each of Examples 3-5 respectively, alongside the thermograms for the raw materials.

FIG. 3 shows DSC curves for Felodipine raw material (solid line) and Felodipine-loaded mesoporous particles (dotted line). A strong endothermic phase transition occurs at 146.3° C. for the raw material, which demonstrates its crystalline nature. No corresponding phase transitions are evident for the Felodipine adsorbed onto the mesoporous particles, showing that it is in amorphous form, which explains the enhanced solubility described below.

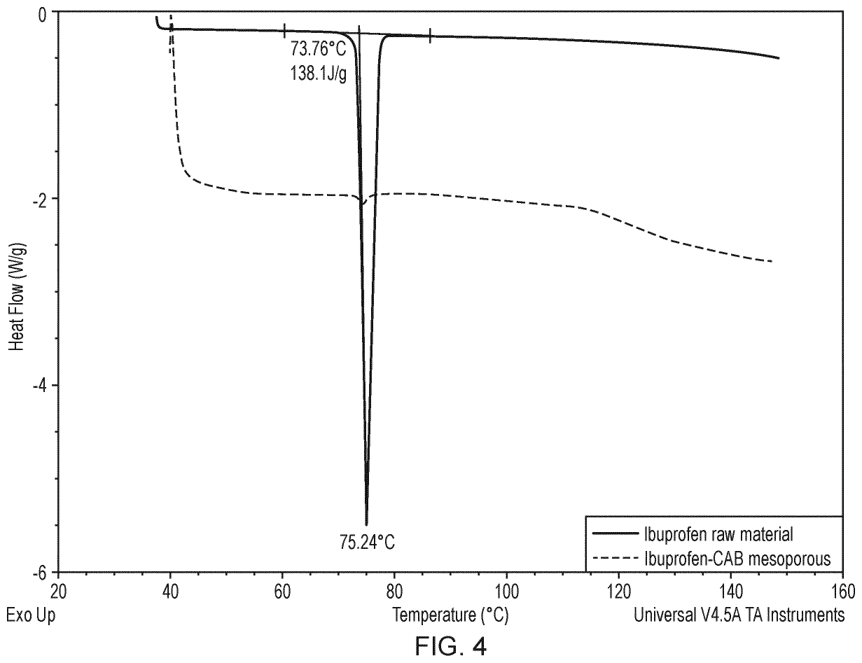

FIG. 4 shows DSC curves for Ibuprofen raw material (solid line) and Ibuprofen-loaded mesoporous particles (dotted line). A strong endothermic phase transition occurs at 75.24° C. for the raw material, which demonstrates its crystalline nature. Only a very weak corresponding phase transition is evident for the Ibuprofen adsorbed onto the mesoporous particles, showing that the majority of the material is in amorphous form, which explains the enhanced solubility described below.

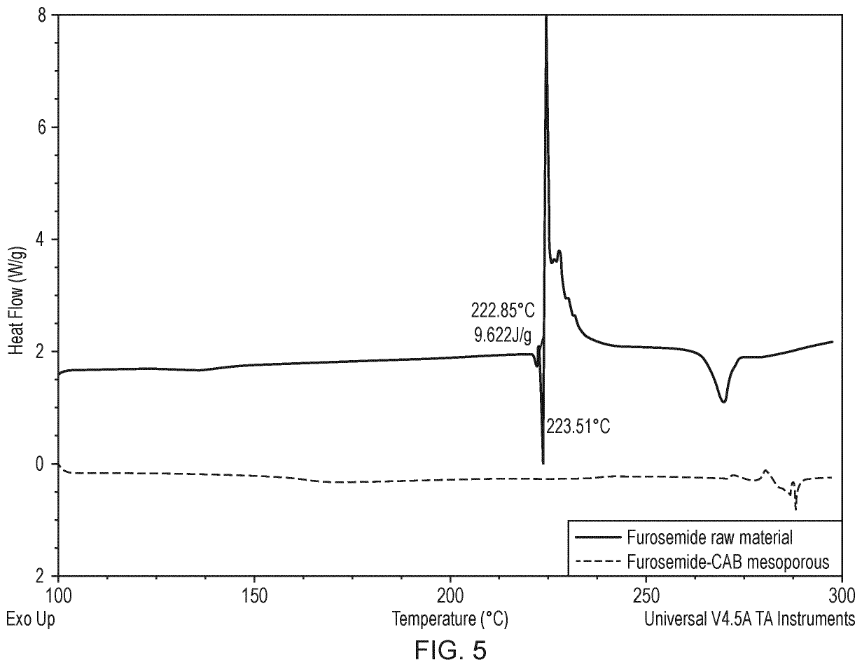

FIG. 5 shows DSC curves for Furosemide raw material (solid line) and Furosemide-loaded mesoporous particles (dotted line). Phase transitions occur at around 220° C. and 265° C. for the raw material, which demonstrates its crystalline nature. No corresponding phase transitions are evident for the Furosemide adsorbed onto the mesoporous particles, showing that it is in amorphous form, which explains the enhanced solubility described below.

### Example 8—Dissolution Profiles of FELO-Loaded Mesoporous Particles

Dissolution testing was performed using USP I apparatus (rotating basket, 50 rpm) in an Erweka DT 126 dissolution tester. Samples of the material as prepared in Example 3 containing 20 mg of FELO were loaded into a HPMC hard-shell capsule and tested in 500 mL of USP pH 6.5 medium with 0.25% sodium lauryl sulfate (SLS) at 37° C. (adapted from USP 36 monograph with a reduction of SLS concentration from 1.0 to 0.25%). Samples were withdrawn during a 120-min period at the following timepoints: 15, 30, 60, 90, and 120 min. The concentrations of dissolved FELO were determined according to a HPLC method described in United States Pharmacopoeia (USP version 36) with mobile phase of USP pH 3 phosphate buffer:acetonitrile:methanol (30:45:25), C18 column (15 cm×4.6 mm, 5 μm), flow rate of 1 mL/min, injection volume of 40 μL, and UV detector at 362 nm in an Agilent 1200 HPLC system.

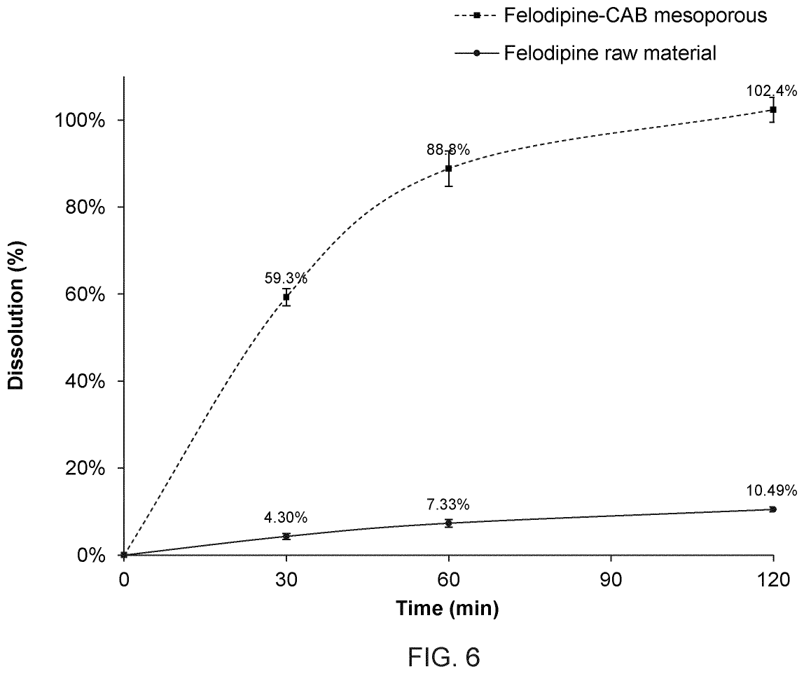

The results are shown in FIG. 6 for the FELO-loaded particles of Example 3 alongside the results for dissolution of the FELO raw material. As can be clearly seen from the plot, the dissolution of Felodipine is greatly enhanced by adsorbing the compound onto the mesoporous particulate material. After 120 mins the dissolution of Felodipine is 10× that seen after the same period for the raw material (i.e. the compound not adsorbed onto any carrier). Indeed, all of the Felodipine adsorbed onto the mesoporous particulate material is fully dissolved after 120 mins, compared with only around 10% of the raw material after the same time period.

### Example 9—Dissolution Profiles of IBU-Loaded Mesoporous Particles

Dissolution testing of IBU-loaded mesoporous particles as prepared in Example 4 was performed by using USP I apparatus (rotating basket, 100 rpm) in an Erweka DT 126 dissolution tester. Samples containing 50 mg of IBU were loaded into a HPMC hard-shell capsule and tested in 900 mL of pH 3.0 medium with 0.25% SLS at 37° C. The pH 3 medium was prepared by dissolving 2 g of sodium chloride and 2.5 g of SLS in 400 mL of deionised water, then adding 0.1 mL of hydrochloric acid 37%, and diluting with deionised water to 1000.0 mL. The concentrations of dissolved IBU were determined using a HPLC method with mobile phase of phosphate buffer pH 3: acetonitrile (60:40), C18 column (15 cm×4.6 mm, 5 μm), flow rate of 2 mL/min, injection volume of 20 μL, and UV detector at 254 nm in an Agilent 1200 HPLC system.

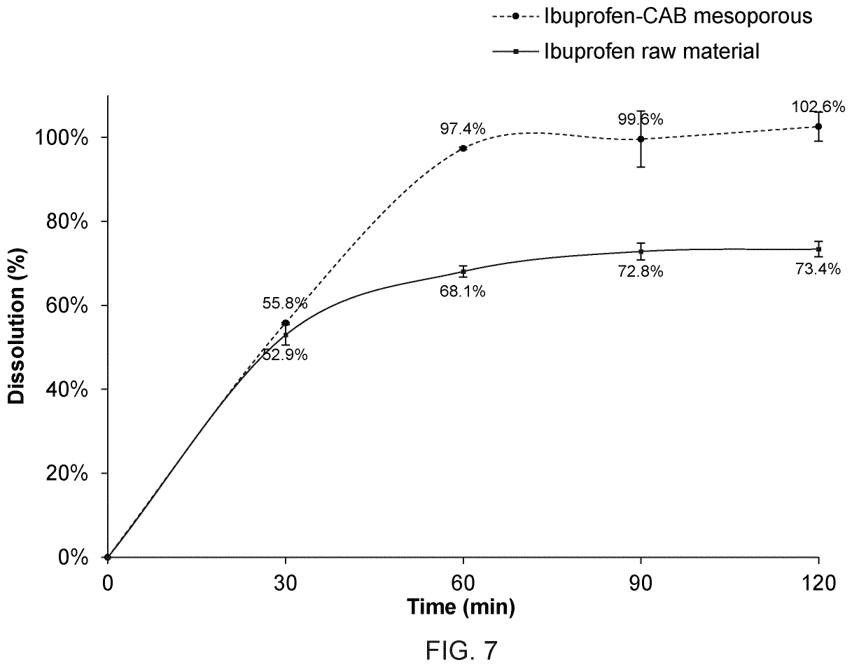

The results are shown in FIG. 7 for the IBU-loaded particles of Example 4 alongside the results for dissolution of the IBU raw material. After 120 mins, all of the Ibuprofen which was adsorbed onto the mesoporous particulate material was dissolved, compared with only 73.4% for the Ibuprofen raw material. Furthermore, a high dissolution rate (97.4%) is achieved for the adsorbed Ibuprofen after a relatively short period of time (60 mins).

### Example 10—Dissolution Profiles of FURO-Loaded Mesoporous Particles

Dissolution testing of FURO-loaded mesoporous particles as prepared in Example 5 was performed by using USP I apparatus (rotating basket, 100 rpm) in an Erweka DT 126 dissolution tester. Samples containing 40 mg FURO were loaded into a HPMC hard-shell capsule and tested in 900 mL of HCl—NaCl pH 3.0 medium with 0.25% SLS at 37° C. The concentrations of dissolved FURO were determined using a HPLC method with mobile phase of phosphate buffer pH 3: acetonitrile (60:40), C18 column (15 cm×4.6 mm, 5 μm), column temperature of 35° C., flow rate of 1 mL/min, injection volume of 10 μL, and UV detector at 234 nm in an Agilent 1200 HPLC system.

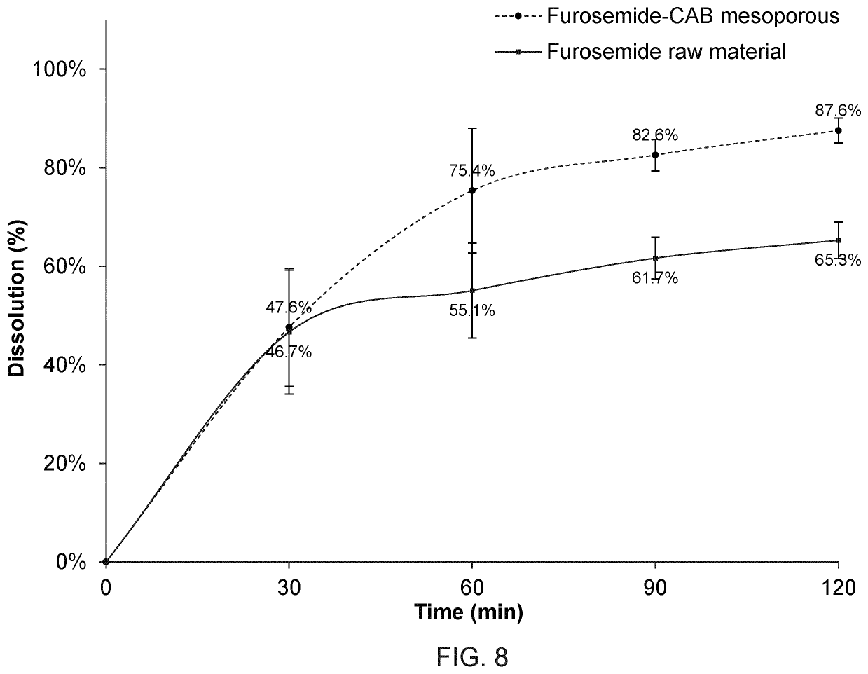

The results are shown in FIG. 8 for the FURO-loaded particles of Example 5 alongside the results for dissolution of the FURO raw material. A significantly higher dissolution rate (87.6%) is achieved for the Furosemide when adsorbed onto the mesoporous particulate material of the invention, compared with only 65.3% for the Furosemide raw material, after 120 mins.

### Example 11—Dissolution Profiles of Co-Spray Dried Felodipine-CAB Polymeric Particles

Dissolution testing of the FELO-loaded mesoporous particles as prepared in Example 6 by the co-spray drying of polymer and Felodipine was performed by using USP I apparatus (rotating basket, 50 rpm) in an Erweka DT 126 dissolution tester. Samples of the material as prepared in Example 6 containing 20 mg of FELO were loaded into a HPMC hard-shell capsule and tested in 500 mL of USP pH 6.5 medium with 0.25% sodium lauryl sulfate (SLS) at 37° C. (adapted from USP 36 monograph with a reduction of SLS concentration from 1.0 to 0.25%). Samples were withdrawn during a 10-hour period at the following timepoints: 0.5, 1, 2, 6, and 10 hours. The concentrations of dissolved FELO were determined according to a HPLC method described in United States Pharmacopoeia (USP version 36) with mobile phase of USP pH 3 phosphate buffer:acetonitrile:methanol (30:45:25), C18 column (15 cm×4.6 mm, 5 μm), flow rate of 1 mL/min, injection volume of 40 μL, and UV detector at 362 nm in an Agilent 1200 HPLC system.

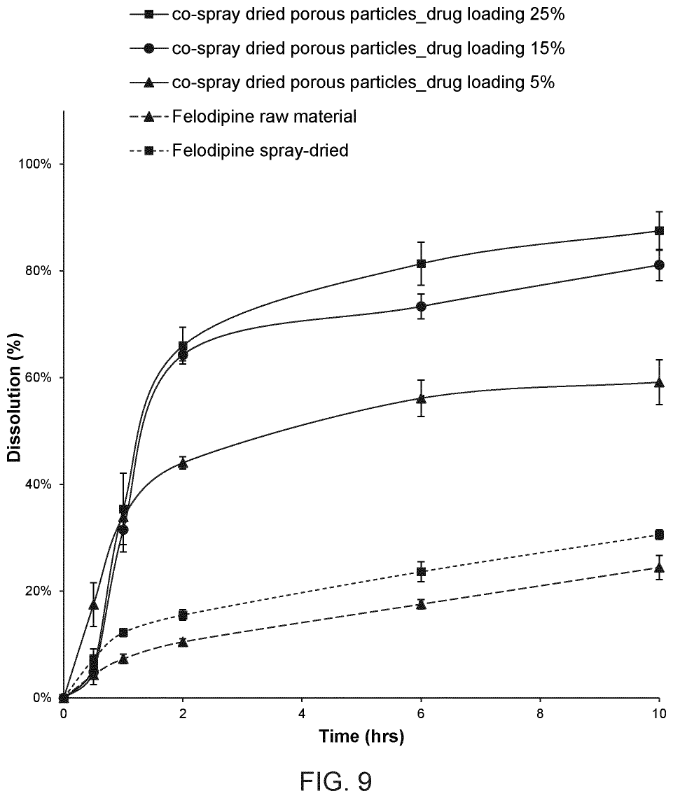

The results are shown in FIG. 9. From the dissolution plots it is evident that both Felodipine raw material and spray-dried raw Felodipine (dotted lines) exhibit poor dissolution, as also evidenced in FIG. 6. By contrast, mesoporous polymeric particles produced by co-spray drying solutions of Felodipine and CAB show much higher dissolution rates after a given period of time, across a range of drug loadings (5%, 15% and 25%). Thus it is evident that the solubility of the compound is enhanced by its loading onto the mesoporous particles.

Additionally, a comparison of FIG. 9 with FIG. 6 reveals that sustained-release properties are imparted on the Felodipine-loaded particles of Example 6 (FIG. 9) relative to those of Example 3 (FIG. 6). When Felodipine is co-spray dried with the polymer, the drug is released more slowly from the particles over an extended period. More specifically, for the 5%, 15% and 25% loaded particles, after 2 hours around 44%, 64% and 66% of the loaded Felodipine had dissolved, respectively, rising to 59%, 81% and 87% respectively after 10 hours. This compares with around 100% dissolution after 2 hours for the post-loaded Felodipine-containing particles of Example 3 (FIG. 6).

### Example 12—Confocal Laser Scanning Microscopy (CLSM) of Mesoporous Particles Loaded with Fluorescein

CLSM was performed on some of the mesoporous particles loaded with a model poorly-soluble compound, fluorescein, to demonstrate the distribution of the model compound.

The mesoporous CAB particles of Sample 8 (Table 2) were post-loaded with fluorescein by following a procedure equivalent to that of Example 3, but substituting fluorescein for felodipine. Mesoporous CAB particles of Sample 8 in Table 2 were added to a solution of Fluorescein (Sigma-Aldrich, analytical reagent) in ethanol (2 mg/mL) to form a suspension at an initial drug load of 20% (w/w). The suspension was gently stirred for 12 h, then spray-dried at inlet temperature of 100° C. using a mini spray dryer Buchi B-290 and inert loop Buchi B-295 in closed mode with nitrogen flow rate of 600 L/min, feed rate of 5 mL/min, and drying gas flow rate of 30 m³/h. These particles post-loaded with fluorescein were denoted Sample 17.

Fluorescein-loaded mesoporous particles were also prepared by co-spray drying. 4.0 g of CAB was mixed with 0.8 g of fluorescein. These mixtures were then each dissolved in 200 mL of acetone:water at ratio of 85:15 (v/v) and co-spray dried using a mini spray dryer Buchi B-290 in closed mode with nitrogen in the Inert Loop Buchi B-295 (Flawil, Switzerland), inlet temperature of 100° C., nitrogen flow rate of 600 L/min, feed rate of 5 mL/min, and drying gas flow rate of 30 m³/h. The spray-dried particles were denoted Sample 18.

The distribution of fluorescein in Samples 17 and 18 was qualitatively evaluated by using Leica confocal microscope TCS SP5 II (Wetzlar, Germany) with 10× and 20× dry objective lens. Excitation and emission wavelength for fluorescein samples were 488 and 525 nm, respectively. Confocal images of fluorescein samples were obtained at 515-535 nm. Scanning depth was 2 μm for both samples with scanning speed was 200 Hz.

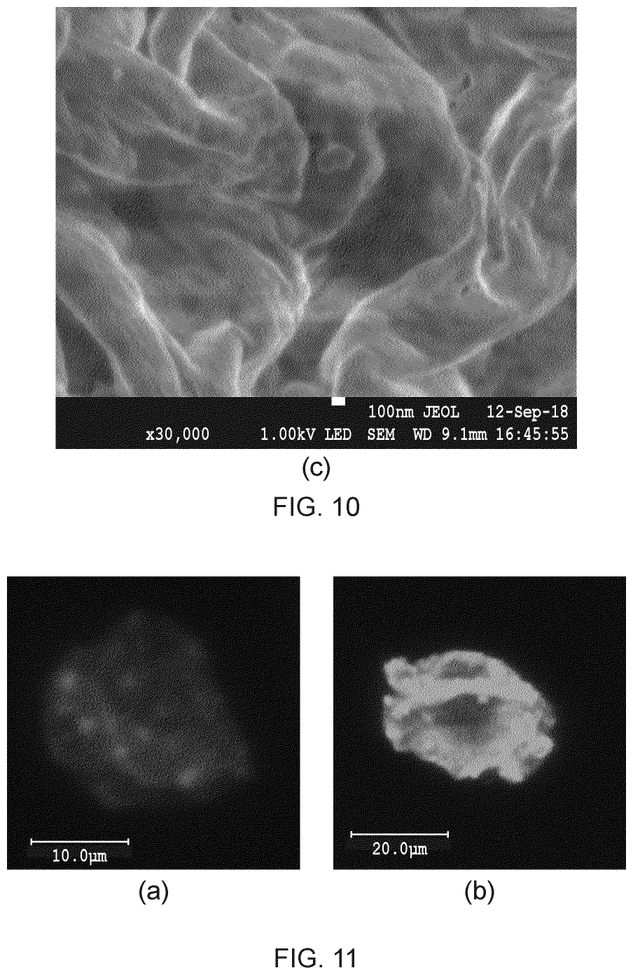

Images obtained from the CLSM are shown in FIG. 11. FIG. 11(a) shows a particle of Sample 17 and FIG. 11(b) shows a particle of Sample 18. The CLSM image of Sample 18 shows that co-spray drying with the poorly soluble compound leads to a distribution of the compound both entrapped within the particles and adsorbed at the particle surface. By contrast, post-loading of particles leads to deposition of the poorly soluble compounds only within the surface pores and the total drug loading is lower.

### Example 13—Determination of Pore Size Distribution

The pore volume and pore size distribution of polymeric mesoporous particles of Sample 8 were analysed by gas adsorption porosimetry using pore size analyser Quantachrome Nova 4200e under the BJH theory according to the method set out in ISO 15901-2 of 2006. Each sample was degassed under vacuum at 100° C. for 24 h before obtaining nitrogen adsorption-desorption measurements.

The results are set out in Table 4 below:

TABLE 4

| | dV/dD | Cumulative Pore | Cumulative Pore |
|---|---|---|---|
| Pore Diameter, | [(cm³/nm/g) × | Volume, V_{cum} | Volume Fraction |
| D (nm) | 10⁻³] | (cm³/g) | (%) |
| 0.0000 | 0.00 | 0.0000 | 0.0% |
| 1.1960 | 0.961 | 0.0002 | 0.1% |
| 1.3222 | 5.08 | 0.0005 | 0.2% |
| 1.4339 | 6.55 | 0.0016 | 0.5% |
| 1.5494 | 7.79 | 0.0021 | 0.7% |
| 1.7745 | 6.65 | 0.0046 | 1.6% |
| 2.0238 | 6.75 | 0.0054 | 1.9% |
| 2.2210 | 6.06 | 0.0071 | 2.4% |
| 2.4822 | 4.79 | 0.0083 | 2.8% |
| 2.7720 | 4.39 | 0.0097 | 3.3% |
| 3.0842 | 3.15 | 0.0106 | 3.7% |
| 3.4220 | 3.01 | 0.0118 | 4.1% |
| 3.8656 | 3.39 | 0.0135 | 4.6% |
| 4.3895 | 2.01 | 0.0146 | 5.0% |
| 4.9998 | 2.33 | 0.0162 | 5.6% |
| 5.7476 | 1.90 | 0.0177 | 6.1% |
| 6.5752 | 2.45 | 0.0198 | 6.8% |
| 8.0070 | 2.49 | 0.0248 | 8.5% |
| 9.8003 | 3.13 | 0.0297 | 10.2% |
| 11.2361 | 3.29 | 0.0340 | 11.7% |
| 12.5497 | 4.05 | 0.0393 | 13.5% |
| 14.3762 | 4.49 | 0.0499 | 17.1% |
| 16.8172 | 5.60 | 0.0641 | 22.0% |
| 19.7873 | 5.18 | 0.0817 | 28.0% |
| 22.8421 | 5.08 | 0.0954 | 32.8% |
| 25.5916 | 5.52 | 0.1108 | 38.0% |
| 29.0798 | 5.57 | 0.1342 | 46.0% |
| 33.9831 | 4.14 | 0.1574 | 54.0% |
| 40.2092 | 4.76 | 0.1900 | 65.2% |
| 50.0931 | 3.08 | 0.2298 | 78.9% |
| 65.6605 | 2.18 | 0.2694 | 92.5% |
| 91.9799 | 0.636 | 0.2913 | 100.0% |

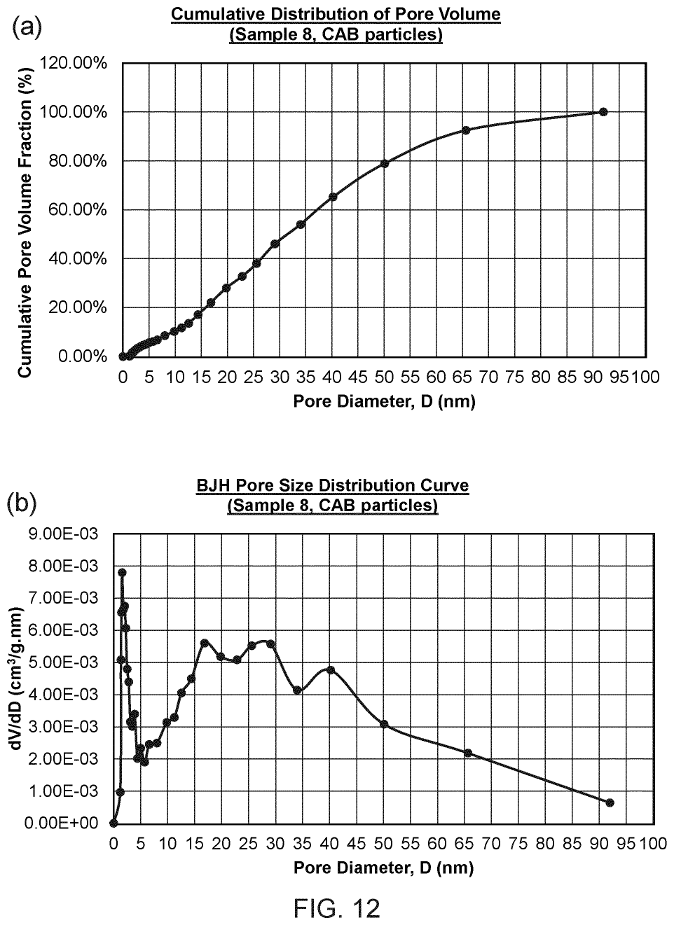

The cumulative distribution of pore volume is plotted in FIG. 12a. The pore size distribution, presented graphically as a plot of dV/dD versus pore size, is shown in FIG. 12b.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the words “have”, “comprise”, and “include”, and variations such as “having”, “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means, for example, +/−10%.

The words “preferred” and “preferably” are used herein refer to embodiments of the invention that may provide certain benefits under some circumstances. It is to be appreciated, however, that other embodiments may also be preferred under the same or different circumstances. The recitation of one or more preferred embodiments therefore does not mean or imply that other embodiments are not useful, and is not intended to exclude other embodiments from the scope of the disclosure, or from the scope of the claims.

1. A particulate material comprising porous polymeric particles, the average pore diameter being from 2 to 50 nm, wherein the porous polymeric particles have a volume mean particle diameter D[4,3] of less than 100 μm and the material is obtained or obtainable by spray-drying a polymer solution.

2. The particulate material according to claim 1, wherein the volume mean particle diameter D[4,3] of the particles is less than 50 μm.

3. The particulate material according to claim 1, wherein the volume of pores in the material is greater than 0.10 cm³/g.

4. The particulate material according to claim 1, wherein the surface area of the material is greater than 10 m²/g.

5. The particulate material according to claim 1, wherein the average pore diameter is from 10 to 30 nm.

6. The particulate material according to claim 1, wherein the particles comprise cellulosic polymer and the polymer solution is a solution comprising the same cellulosic polymer.

7. The particulate material according to claim 6, wherein the cellulosic polymer is selected from one or more of cellulose esters and cellulose ethers.

8. The particulate material according to claim 6, wherein the cellulosic polymer is selected from one or more of cellulose acetate butyrate, cellulose acetate, methyl cellulose, ethyl cellulose, hydroxyethyl cellulose, hydroxypropyl cellulose, carboxymethyl cellulose and hydroxypropyl methyl cellulose.

9. The particulate material according to claim 8, wherein the cellulosic polymer is cellulose acetate butyrate.

10. The particulate material according to claim 1, wherein the inlet temperature during spray-drying of the polymer solution is lower than the glass transition temperature T_{g} of the polymer in the polymer solution.

11. The particulate material according to claim 1, wherein the glass transition temperature T_{g} of the polymer in the polymer solution is greater than 100° C.

12. The particulate material according to claim 1, wherein the solution comprises a solvent mixture comprising water and acetone.

13. A pharmaceutical composition comprising a particulate material according to claim 1 loaded with one or more active pharmaceutical compounds.

14. (canceled)

15. A method of treatment of the human or animal body, comprising administration of a therapeutically effective amount of the pharmaceutical composition according to claim 13.

16. A method of manufacturing a particulate material comprising spray-drying a polymer solution, the particulate material comprising porous polymeric particles, the average pore diameter being from 2 to 50 nm, wherein the porous polymeric particles have a volume mean diameter D[4,3] of less than 100 μm.

17. The method according to claim 16, wherein the polymer solution is a solution comprising cellulosic polymer.

18. The method according to claim 17, wherein the cellulosic polymer is selected from one or more of cellulose esters and cellulose ethers.

19. The method according to claim 17, wherein the cellulosic polymer is selected from one or more of cellulose acetate butyrate, cellulose acetate, methyl cellulose, ethyl cellulose, hydroxyethyl cellulose, hydroxypropyl cellulose, carboxymethyl cellulose and hydroxypropyl methyl cellulose.

20. The method according to claim 19, wherein the cellulosic polymer is cellulose acetate butyrate.

21. The method according to claim 16, wherein the inlet temperature during spray-drying of the polymer solution is lower than the glass transition temperature T_{g} of the polymer in the polymer solution.

22. The method according to claim 16, wherein the glass transition temperature T_{g} of the polymer in the polymer solution is greater than 100° C.

23. The method according to claim 16, wherein the solution comprises a solvent mixture comprising water and acetone.

24. The method according to claim 16, wherein the solution comprises one or more active pharmaceutical compounds.

25. The method according to claim 16, wherein the spray-drying is carried out in a spray-dryer under closed-mode with nitrogen, an inlet temperature of from 60 to 180° C. and an atomisation pressure of from 100 to 500 KPa.

26. Use of a particulate material according to claim 1 as a solubility-enhancing carrier for one or more active pharmaceutical compounds.
